(19) United States
(12) Patent Application Publication
(10) Pub. No.: US 2022/0062493 A1
(43) Pub. Date: Mar. 3, 2022

(54) ALKYLAMINE FUNCTIONALISED SUBSTRATE FOR DELIVERING CELLS TO A SITE AND METHODS OF USE OF THE FUNCTIONALISED SUBSTRATE

(71) Applicant: TekCyte Pty Ltd, Adelaide (AU)

(72) Inventors: Louise Elizabeth SMITH, Mawson Lakes (AU); Andrew Percival MICHELMORE, Teringie (AU); Giles Thomas Sipho KIRBY, Mawson Lakes (AU); Allison June COWIN, St. Georges (AU); Stuart James MILLS, Collinswood (AU); Robert David SHORT, Burnside (AU)

(21) Appl. No.: 17/521,607

(22) Filed: Nov. 8, 2021

Related U.S. Application Data
(62) Division of application No. 15/551,567, filed on Aug. 16, 2017.

(30) Foreign Application Priority Data
Feb. 16, 2015 (AU) 2015900510

(51) Int. Cl. A61L 15/40; A61L 15/26; A61K 35/28; A61L 15/44

(52) U.S. Cl. CPC A61L 15/40; A61L 15/26; A61K 35/28; A61L 2300/64; A61L 2420/02; A61L 2400/18; A61L 15/44

(57) ABSTRACT

The present disclosure relates to methods and products for delivering cells to a biological site. Certain embodiments of the present disclosure provide a method of delivering cells to a biological site. The method comprises providing a product comprising an alkylamine functionalised substrate and cells for delivery to the biological site attached to the functionalised substrate, wherein the alkylamine functionalised substrate comprises a surface density with an atomic ratio of primary amine to carbon of greater than 0.005, and applying the product to the biological site to allow transfer of the cells from the product to the biological site, thereby delivering cells to the site.

### CROSS-REFERENCE TO RELATED APPLICATIONS

This application is a continuation of U.S. patent application Ser. No. 15/551,567, filed Aug. 16, 2017, which is a National Stage Application of International Patent Application PCT/AU2016/050101, which claims priority to Australian Provisional Patent Application No. 2015900510, filed on Feb. 16, 2015, the contents of which are hereby incorporated by reference.

### FIELD

The present disclosure relates to methods and products for delivering cells to a biological site.

### BACKGROUND

The ability to deliver cells to a desired site provides a possible therapeutic avenue for a variety of diseases, conditions and states. For example, the ability to deliver stem cells has promising therapeutic potential for some degenerative diseases, such as the delivery of stem cells to the heart to treat congestive heart failure or the delivery of stem cells for the treatment of neurodegenerative conditions. The cells to be delivered can have therapeutic potential in their own right, and/or be used as vehicles to deliver therapeutic agents to desired sites.

The healing of wounds is another example where the delivery of cells has therapeutic potential. Despite advances in the understanding of the principles underlying the wound healing process, the therapeutic options for wound treatment still remain limited. Cell delivery strategies provide a potential therapeutic avenue.

Wounds can result from a variety of causes, including for example trauma, disease, action of micro-organisms and exposure to foreign materials. Wound healing is not only important to achieve wound closure, but is also important to restore tissue functionality and to provide a barrier function against infection. Delayed wound healing is a significant contributor to morbidity in subjects. In some situations, the wound healing process is dysfunctional, leading to the development of chronic wounds. Chronic wounds have major impacts on the physical and mental health, productivity, morbidity, mortality and cost of care for affected individuals.

The most commonly used conventional modality to assist in wound healing involves the use of wound dressings. A variety of different types of dressings are used to assist with wound healing. Some treatments have also utilized the provision of minerals and vitamins to assist with wound healing. However, these types of treatment modalities have met with little success. As such, current clinical approaches used to promote wound healing include protection of the wound bed from mechanical trauma, control of surface microbial burden through antibiotics, antiseptics and other antimicrobial compounds, and the use of some types of growth factors. However, these approaches all have a variety of disadvantages.

Whilst the delivery of cells has therapeutic potential, the use of cell delivery still remains limited for a number of reasons. For example, considerations such as how cells should be delivered, substrate selection, attachment of cells, efficiency of cell transfer and/or the ability of cells to retain their therapeutic properties are important to therapeutic outcome.

Accordingly, there is a continuing need to identify new means for delivery of cells, including for therapeutic purposes.

### SUMMARY

The present disclosure relates to methods and products for delivering cells to a site.

Certain embodiments of the present disclosure provide a method of delivering cells to a biological site, the method comprising:
providing a product comprising an alkylamine functionalised substrate and cells for delivery to the biological site attached to the functionalised substrate, wherein the alkylamine functionalised substrate comprises a surface density with an atomic ratio of primary amine to carbon of greater than 0.005; and applying the product to the biological site to allow transfer of the cells from the product to the biological site;
thereby delivering cells to the site.

Certain embodiments of the present disclosure provide a method of delivering cells to a wound, the method comprising:
providing a wound healing product comprising an alkylamine functionalised substrate and cells for delivery to the wound attached to the functionalised substrate, wherein the alkylamine functionalised substrate comprises a surface density with an atomic ratio of primary amine to carbon of greater than 0.005; and
applying the product to the wound to allow transfer of the cells from the product to the wound;
thereby delivering cells from the wound healing product to the wound.

Certain embodiments of the present disclosure provide a product for delivering cells to a biological site, the product comprising an alkylamine functionalised substrate and cells for delivery to the site attached to the functionalised substrate, wherein the alkylamine functionalised substrate comprises a surface density with an atomic ratio of primary amine to carbon of greater than 0.005.

Certain embodiments of the present disclosure provide a wound healing product comprising an alkylamine functionalised substrate and cells for healing a wound attached to the functionalised substrate, wherein the the alkylamine functionalised substrate comprises a surface density with an atomic ratio of primary amine to carbon of greater than 0.005.

Certain embodiments of the present disclosure provide a wound healing product comprising:
an alkylamine functionalised substrate, wherein the alkylamine functionalised substrate comprises a surface density with an atomic ratio of primary amine to carbon of greater than 0.005; and
cells for healing a wound attached to the substrate.

Certain embodiments of the present disclosure provide a composition comprising an alkylamine functionalised substrate and cells for healing a wound attached to the substrate, wherein the alkylamine functionalised substrate comprises a surface density with an atomic ratio of primary amine to carbon of greater than 0.005.

Certain embodiments of the present disclosure provide a method of treating a wound, the method comprising applying to the wound a product comprising an alkylamine functionalised substrate and cells for healing the wound attached to the functionalised substrate, wherein the alkylamine functionalised substrate comprises a surface density with an atomic ratio of primary amine to carbon of greater than 0.005.

Certain embodiments of the present disclosure provide a method of producing a wound healing product comprising cells for healing a wound attached to a substrate, the method comprising attaching the cells for healing the wound to the substrate which has been functionalised with an alkylamine and comprises a surface density with an atomic ratio of primary amine to carbon of greater than 0.005.

Certain embodiments of the present disclosure provide a method of producing a wound healing product comprising cells for healing a wound attached to a substrate, the method comprising:
functionalising the substrate with a plasma polymerised alkylamine, wherein the functionalising of the substrate produces a substrate with a surface density with an atomic ratio of primary amine to carbon of greater than 0.005; and attaching the cells for healing a wound to the functionalised substrate.

Certain embodiments of the present disclosure provide a method of modifying a substrate for attachment of cells, the method comprising exposing the substrate to plasma polymerisation with an alkylamine to modify the substrate, wherein the plasma polymerization with the alkylamine produces a substrate with a surface density with an atomic ratio of primary amine to carbon of greater than 0.005.

Certain embodiments of the present disclosure provide a method of functionalising a substrate for attachment of cells, the method comprising modifying the substrate by plasma polymerisation with an alkylamine to functionalise the substrate, wherein the plasma polymerization with the alkylamine produces a surface density with an atomic ratio of primary amine to carbon of greater than 0.005.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows that cultured MAPCs exhibit appropriate and expected morphology on fibronectin coated tissue culture plastic. The doubling times observed were as expected. The left panel shows MAPCs immediately after seeding on the plate, while the right panel shows MAPCs after expansion.

FIG. 2 shows real time PCR of a selection of key markers in MAPCs and donor matched MSCs which indicates that the MAPCs are within pre-defined tolerances, confirming that the cells are MAPCs.

FIG. 3 shows transfer assay in vitro with metabolic activity quantified using MTT reagent. Lower power levels were more favourable for cell transfer. Patches with a 5 W acid plasma polymerisation were able to deliver cells to the dermis with a metabolic activity approximately 80% that of TCP. All of the conditions were with a monomer flow rate of 4 sccm.

FIG. 4 shows images of MTT stained silicone and dermis. Purple colour indicates metabolising cells.

FIG. 5 shows that initial screening with allylamine was less favourable than heptylamine, with a 5 W heptylamine plasma polymer able to deliver cells to a model wound site with an equal metabolic activity to that of cells grown on fibronectin coated TCP. All of these conditions were with a monomer flow rate of 4 sccm.

FIG. 6 shows a comparison of heptylamine flow rates indicating that a flow rate in the range of 4 sccm was favourable.

FIG. 7 shows images of MTT stained silicone and dermis in transfer experiments using heptylamine functionalised substrates. Purple colour indicates metabolising cells.

FIG. 8 shows real time PCR of a selection of key markers in MAPCs and donor matched MSCs, which indicates that MAPCs cultured for 48 hours on a candidate surface are within pre-defined tolerances, confirming they are MAPCs.

FIG. 9 shows XPS spectra of silicone coated with a 5 W acrylic acid plasma polymer.

FIG. 10 shows XPS spectra of silicone coated with a 5 W propanoic acid plasma polymer.

FIG. 11 shows a XPS spectra of silicone coated with a 5 W allylamine plasma polymer.

FIG. 12 shows a XPS spectra of silicone coated with a 5 W heptylamine plasma polymer.

FIG. 13 shows relative percentages of silicon measured in candidate patches over a 12 day time course. An upward trend can be seen as the levels of silicon increase over time in all patches.

FIG. 14 shows functionality of plasma polymer surfaces in response to changing RF power.

FIG. 15 shows images of MAPCs transferred to dermis and cultured upon HaPP silicone patches prepared at a variety of powers. Cell locations are indicated by the purple MTT formazan product of metabolic activity. Dermis indicates the cells transferred onto dermis and NT indicates cells that were Not Transferred and simply cultured on the surface. The positive control are MAPCs cultured upon fibronectin coated TCP.

FIG. 16 shows quantification of the MTT formazan product from the dermis shown in FIG. 15 as well as the positive control, MAPCs cultured upon fibronectin coated TCP.

FIG. 17 shows quantification of the MTT formazan product from the non-transferred cells shown in FIG. 15, which shows that at lower powers cells are more metabolically active.

FIG. 18 shows primary amine as a ratio of nitrogen as a function of power for the heptylamine functionalised substrate.

FIG. 19 shows primary amine as a ratio of carbon as a function of power for the heptylamine functionalised substrate.

FIG. 20 shows the data from FIG. 19 plotted against a logarithmic scale.

FIG. 21 shows primary amine ratio versus cell transfer ability.

FIG. 22 shows primary amine ratio versus cell culture ability (cells not transferred).

FIG. 23 shows the results of cell transfer studies using heptylamine, diaminoproapane or octadiene functionalised substrate as a function of the primary amine to carbon ratio.

FIG. 24 shows images of 6-well plates showing the blue/purple insoluble formazan product resulting from metabolically active cells. The plasma polymer coated IV3000 was effective for the transfer of MAPCs. Plasma polymer coated Melolin was less effective. Both were suitable for the culture of MAPCs.

FIG. 25 shows quantification of MTT-Formazan product from the transfer of MAPCs from PP treated Melolin and IV3000.

FIG. 26 shows DED imaged following the delivery of fibroblasts isolated from three separate donors and stained using MTT for metabolic activity.

FIG. 27 shows DED imaged following the delivery of keratinocytes and stained using MTT for metabolic activity. (A) was cultured in Greens medium (high calcium and 10% serum), (B) and (C) were cultured in low calcium, serum free conditions These samples were cultured in serum free (SF) media with the potential that weaker cell-cell binding may lead to a greater cell delivery.

FIG. 28 shows quantification of MTT-Formazan product from the transfer of Fibroblasts and Keratinocytes. Data is normalised to a control cultured in tissue culture well plates.

FIG. 29 shows macroscopic measurements showing effect of cells (MAPCs) delivered by the HaPP-medical grade silicone patch at different dosages in diabetic mouse wounds. A) Percentage of original wound area normalized to day 0 wound measurements in diabetic mice, and B) a bar chart illustrating the percentage of original wound area results at D3 and D7.

FIG. 30 shows macroscopic measurements showing effect of MAPCs delivered by the HaPP-medical grade silicone patch vs injection in acute mouse wounds. A) wound gape, and B) percentage of original wound area normalized to day 0 wound measurements in acute mice wounds. In A and B, cell injection is the control.

FIG. 31 shows macroscopic measurements showing effect of cells delivered by the HaPP-medical grade silicone patch vs injection in diabetic mouse wounds. A) wound gape, and B) percentage of original wound area normalized to day 0 wound measurements in diabetic mice. In A and B, the HaPP-medical grade silicone patch (without cells) is the control. Microscopic measurements showing effect of cells delivered by the HaPP-medical grade silicone patch vs HaPP-medical grade silicone patch (without cells) in diabetic mouse wounds C) wound width, D) percentage reepithelialisation and E) wound area measurements.

FIG. 32 shows cells delivered by HaPP-medical grade silicone patch increase reepithelialisation of diabetic mouse wounds. Representative macroscopic images for A) day 3 and B) day 7 wounds treated with cell injection, HaPP-medical grade silicone alone and HaPP-medical grade silicone with 20×10³ cells. The black lines demarcate the unepithelialized areas of the wounds.

FIG. 33 shows identification of cells within d3 and d7 mouse wounds treated with 2×10³ cells delivered using the HaPP-medical grade silicone patch. A human nuclear antigen detects the human cells (MAPCs) and the wounds are counterstained with DAPI (blue).

### DETAILED DESCRIPTION

The present disclosure relates to methods and products for delivering cells to a biological site.

Certain embodiments of the present disclosure provide a method of delivering cells to a biological site.

Certain embodiments of the present disclosure provide a method of delivering cells to a biological site, the method comprising:
providing a product comprising an alkylamine functionalised substrate and cells for delivery to the biological site attached to the functionalised substrate, wherein the alkylamine functionalised substrate comprises a surface density with an atomic ratio of primary amine to carbon of greater than 0.005; and applying the product to the biological site to allow transfer of the cells from the product to the biological site;
thereby delivering cells to the biological site.

Examples of biological sites include a site for tissue or cell repair, a site for tissue or cell production, a site for tissue or cell regeneration, a site benefiting from the delivery of cells, such cartilage, bone, fat and/or a site of neovascularisation. Examples of other sites include cutaneous wounds, both acute and chronic, sites of ocular injury (such as the cornea), heart tissue and the surface of an organ. Chronic wounds include neuropathic ulcers, diabetic ulcers, ischemic ulcers, pressure ulcers, or wounds caused by dehiscence. Cutaneous wounds also include burns and scalds. Other types of sites of action are contemplated.

For example, the product (such as a patch) could be used to treat ocular injuries where therapeutic cells are delivered to the eye to resurface the cornea or similar.

In certain embodiments, the cells comprise multipotent cells.

In certain embodiments, the cells comprise stem cells, such as adult/somatic stem cells. In certain embodiments, the cells comprise multipotent stem cells capable of differentiating to form adipocytes, cartilage, bone, tendons, muscle, and skin.

In certain embodiments, the cells comprise multipotent adult progenitor cells (MAPCs).

The term “multipotent adult progenitor cells” or “MAPCs” as used herein is to be understood to mean cells usually isolated from bone marrow and which are significantly smaller than mesenchymal stem cells (Sohni A. and Verfaillie C. M. (2011) “Multipotent adult progenitor cells” Best Pract Res Clin Haematol. 24(1): 3-11); Verfaillie C. M. and Crabbe A. (2009) in “Essentials of Stem Cell Biology” ed. Robert Lanza et. al. Al. Elsevier Inc).

MAPCs proliferate without senescence and have a broad differentiation ability (Reyes M. et al. (2001) “Purification and ex vivo expansion of postnatal human marrow mesodermal progenitor cells” Blood 98(9): 2615-25; Jiang et al (2002) “Pluripotency of mesenchymal stem cells derived from adult marrow” Nature 418(6893):41-90).

MAPCs may be expanded in vitro for greater than 70 population doublings, more than equivalent human MSCs (20-25 doublings) (Roobrouck et al. (2011) “Differentiation potential of human postnatal mesenchymal stem cells, mesoangioblasts, and multipotent adult progenitor cells reflected in their transcriptome and partially influenced by the culture conditions” Stem Cells 29(5):871-82).

hMAPCs and hMSCs are two distinct cell populations. In contrast to hMSCs, hMAPCs are negative for CD140a, CD140b and alkaline phosphatase, and express low levels of MHC class 1 (Jacobs et al (2013) “Human multipotent adult progenitor cells are nonimmunogenic and exert potent immunomodulatory effects on alloreactive T-cell responses” Cell Transplant. 22(10):1915-28); Jacobs et al. (2013) “Immunological characteristics of human mesenchymal stem cells and multipotent adult progenitor cells” Immunol Cell Biol. 2013 91(1):32-9).

In certain embodiments, the cells comprise multipotent stromal cells.

In certain embodiments, the cells comprise mesenchymal stem cells (MSCs). Mesenchymal stem cells have the potential to differentiate towards lineages of mesenchymal origin, including bone, cartilage, fat, connective tissue, smooth muscle and hematopoietic supportive stroma and may be isolated from bone marrow, adipose tissue, synovial fluid, periosteum, umbilical cord blood and some fetal tissues (Pittenger M. F. et al. (1999) “Multilineage potential of adult human mesenchymal stem cells” Science 284: 143-147; Bieback K. et al. (2004) “Critical parameters for the isolation of mesenchymal stem cells from umbilical cord blood” Stem Cells 22: 625-634; De Bari C. et al. (2001) “Human periosteum-derived cells maintain phenotypic stability and chondrogenic potential throughout expansion regardless of donor age” Arthritis Rheum 44: 85-95; In't Anker P. S. et al. (2003) “Amniotic fluid as a novel source of mesenchymal stem cells for therapeutic transplantation” Blood 102: 1548-1549; Zuk P. A. et al. (2002) “Human adipose tissue is a source of multipotent stem cells” Mol Biol Cell 13: 4279-4295).

Methods for isolating cells, including multipotent adult progenitor cells and mesenchymal stem cells, are known in the art.

In certain embodiments, the cells comprises bone marrow derived mononuclear cells, adherent stromal cells including mesenchymal stem cells (isolated from sources including bone marrow, adipose tissue, skin, blood or other human tissues or fluids), hematopoetic stem cells, endothelial progenitor cells and other progenitor cells, fibroblasts, keratinocytes, endothelial cells, melanocytes. Other types of cells are contemplated.

In certain embodiments, the attaching of cells to the functionalised substrate comprises passive attachment of the cells to the substrate. For example, cells may be placed and/or cultured in the presence of the substrate and attachment of the cells obtained in this way. Other methods for attachment of the cells to the substrate are contemplated.

In certain embodiments, applying the product to the site to allow transfer of the cells from the product to the site is achieved by placing the product in direct contact with the site. For example, a wound healing product may be placed in directed contact with the wound.

In certain embodiments, applying the product to the site to allow transfer of the cells from the product to the site is achieved by indirect contact with the site, and allowing migration of the cells to the desired site. For example, a composition comprising the functionalised substrate and cells attached to the substrate may be administered to a subject and cells released from the product can move to a remote site of action. For example, a composition comprising particles could be delivered by implantation into a subject and cells transferred to a desired site of action by migration of the cells from the site of implantation to the desired site of action.

In certain embodiments, the product comprises a degradable carrier. For example, a patch having a degradable carrier may be used internally to deliver cells to the surface of an organ.

In certain embodiments, the site comprises a wound. The term “wound” includes for example an injury to a tissue, including open wounds, delayed or difficult to heal wounds, and chronic wounds. Examples of wounds may include both open and closed wounds. The term “wound” also includes, for example, injuries to the skin and subcutaneous tissue and injuries initiated in different ways and with varying characteristics .

In certain embodiments, the wound comprises an external wound. In certain embodiments, the wound comprises an open wound. In certain embodiments, the wound comprises a chronic wound. In certain embodiments, the wound comprises a chronic wound or an ulcer, such as a diabetic wound or a diabetic ulcer.

For external wounds, typically these wounds are classified into one of four grades depending on the depth of the wound: i) Grade I wounds limited to the epithelium; ii) Grade II wounds extending into the dermis; iii) Grade III wounds are full thickness wounds or wounds extending into the subcutaneous tissue; and iv) Grade IV wounds are wounds where bones are exposed.

In certain embodiments, the alkylamine functionalised substrate comprises a substrate functionalised with a mono-amino alkane. In certain embodiments, the alkylamine functionalised substrate comprises a substrate functionalised with a di-amino alkane.

In certain embodiments, the alkylamine functionalised substrate comprises a substrate functionalised with one or more of ammonia, methylamine, ethylamine, propylamine, isopropylamine, allylamine, n-butylamine, tert-butylamine, sec-butylamine, isobutylamine, pentylamine, hexylamine, heptylamine, ethylenediamine, 1,2-diaminopropane, 1,3-diaminopropane, 1,4-diaminobutane, 1,5-diaminopentane, cycloaminopropane, (methane/ammonia mixtures), (ethylene/ammonia mixtures), substituted derivatives of any of the aforementioned, copolymers of any of the aforementioned, and copolymers of one or more of the aformentioned with a hydrocarbon (eg an alkane, alkene, alkyne). Other alkylamine functionalised substrates are contemplated. Methods for functionalising a substrate with an alkylamine are described herein

In certain embodiments, the alkylamine functionalised substrate comprises a substrate functionalised with heptylamine and/or a substituted derivative thereof.

In certain embodiments, the substrate comprises a silicone and/or a polyurethane.

Other examples of a substrate include synthetic or natural polymers, including polymers that can be formed into sheets or thin fibres, copolymers or blends of polymers such as nylons, polyesters, polyethylenes, polyethylene terephthalate, elastomers such as silicones and polydimethylsiloxane, polyurethanes, polycaprolactone, copolymers and blends of the aforementioned, degradable polymers and polycaprolactone, poly lactic acid and polyglycolic acid, including copolymers and blends, polyhydroxybutyrate and polyhydroxyvalerate and copolymers and blends, silk, nylon polymers, nylon 66 polymers, polyethylene polymers, polypropylene polymers, poly(tetrafluoroethylene) (PTFE) polymers, poly(vinylidene fluoride) (PVDF) polymers, viscose rayon polymers, polycaprolactone polymers, polydioxanone polymers, polygalctin polymers, poly(glycolide-co-caprolactone) polymers, and poly(glycolide-trimethylene carbonate polymers. Other types of substrates are contemplated.

In certain embodiments, the substrate comprises one or more polymers.

In certain embodiments, the substrate is a non-metal substrate.

In certain embodiments, the product comprises a bandage, a gauze, a patch or a dressing.

In certain embodiments, the product comprises an implantable product. In certain embodiments, the product comprises a composition. In certain embodiments, the product comprises particles or beads. Methods for producing a product comprising an alkylamine functionalised substrate are known in the art.

In certain embodiments, the surface density of the functionalised substrate comprises an atomic ratio of primary amine to carbon of greater than 0.006, greater than 0.007, greater than 0.008 or greater than 0.009.

In certain embodiments, the surface density of the functionalised substrate comprises an atomic ratio of primary amine to carbon of greater than 0.009.

In certain embodiments, the surface density comprises an atomic ratio of primary amine to carbon in the range from 0.005 to 0.04, 0.005 to 0.035, 0.005 to 0.03, 0.005 to 0.025, 0.005 to 0.02, 0.005 to 0.015, 0.005 to 0.01, 0.005 to 0.009, 0.005 to 0.008, 0.005 to 0.007, and 0.005 to 0.006.

In certain embodiments, the surface density comprises an atomic ratio of primary amine to carbon in the range from 0.009 to 0.04, 0.009 to 0.035, 0.009 to 0.03, 0.009 to 0.025, 0.009 to 0.02, 0.009 to 0.015 and 0.009 to 0.010.

In certain embodiments, the surface density comprises an atomic ratio of primary amine to carbon in the range from 0.009 to 0.04.

Methods for determining surface density are known in the art.

In certain embodiments, the surface density comprises an atomic ratio of primary amine to nitrogen of greater than 0.08, greater than 0.09, greater than 0.10, greater than 0.011, greater than 0.12, greater than 0.13, greater than 0.14, greater than 0.15, greater than 0.16, greater than 0.17, greater than 0.18 or greater than 0.19.

In certain embodiments, the surface density comprises an atomic ratio of primary amine to nitrogen of greater than 0.08.

In certain embodiments, the surface density comprises an atomic ratio of primary amine to nitrogen in the range from 0.08 to 0.20, 0.08 to 0.19, 0.08 to 0.18, 0.08 to 0.17, 0.08 to 0.16, 0.08 to 0.15, 0.08 to 0.14, 0.08 to 0.13, 0.08 to 0.12, 0.08 to 0.11, 0.08 to 0.10, 0.08 to 0.09, 0.09 to 0.20, 0.09 to 0.19, 0.09 to 0.18, 0.09 to 0.17, 0.09 to 0.16, 0.09 to 0.15, 0.09 to 0.14, 0.09 to 0.13, 0.09 to 0.12, 0.09 to 0.11, 0.09 to 0.10, 0.10 to 0.20, 0.10 to 0.19, 0.10 to 0.18, 0.10 to 0.17, 0.10 to 0.16, 0.10 to 0.15, 0.10 to 0.14, 0.10 to 0.13, 0.10 to 0.12, 0.10 to 0.11, 0.11 to 0.20, 0.11 to 0.19, 0.11 to 0.18, 0.11 to 0.17, 0.11 to 0.16, 0.11 to 0.15, 0.11 to 0.14, 0.11 to 0.13, 0.11 to 0.12, 0.12 to 0.20, 0.12 to 0.19, 0.12 to 0.18, 0.12 to 0.17, 0.12 to 0.16, 0.12 to 0.15, 0.12 to 0.14, 0.12 to 0.13, 0.13 to 0.20, 0.13 to 0.19, 0.13 to 0.18, 0.13 to 0.17, 0.13 to 0.16, 0.13 to 0.15, 0.13 to 0.14, 0.14 to 0.20, 0.14 to 0.19, 0.14 to 0.18, 0.14 to 0.17, 0.14 to 0.16, 0.14 to 0.15, 0.15 to 0.20, 0.15 to 0.19, 0.15 to 18, 0.15 to 0.17, 0.15 to 0.16, 0.16 to 0.20, 0.16 to 0.19, 0.16 to 0.18, 0.16 to 0.17, 0.17 to 0.20, 0.17 to 0.19, 0.17 to 0.18, 0.18 to 0.20, 0.18 to 0.19, and 0.19 to 0.20.

In certain embodiments, the surface density comprises an atomic ratio of primary amine to nitrogen ratio in the range from 0.08 to 0.20.

In certain embodiments, the functionalisation of the substrate comprises plasma polymerisation with the alkylamine at a power of 20 W or less, 15 W or less, 10 W or less, or 2W or less.

In certain embodiments, the functionalisation of the substrate comprises plasma polymerisation with the alkylamine at a power of 20 W or less. In certain embodiments, the functionalisation of the substrate comprises plasma polymerisation with the alkylamine at a power of 10 W or less.

In certain embodiments, the functionalisation of the substrate comprises plasma polymerisation with the alkylamine at a power in the range from 1 W to 10 W.

In certain embodiments, the functionalisation of the substrate comprises plasma polymerisation with the alkylamine at a flow rate of greater than 1 sccm (standard cubic centimetres per minute). In certain embodiments, the functionalisation of the substrate comprises plasma polymerisation with the alkylamine at a flow rate of greater than 2 sccm. In certain embodiments, the functionalisation of the substrate comprises plasma polymerisation with the alkylamine at a flow rate of greater than 3 sccm. In certain embodiments, the functionalisation of the substrate comprises plasma polymerisation with the alkylamine at a flow rate of greater than 4 sccm. In certain embodiments, the functionalisation of the substrate comprises plasma polymerisation with the alkylamine at a flow rate of greater than 5 sccm.

In certain embodiments, the functionalisation of the substrate comprises plasma polymerisation with the alkylamine at a flow rate in the range of 1 to 10 sccm. In certain embodiments, the functionalisation of the substrate comprises plasma polymerisation with the alkylamine at a flow rate in the range of 1 to 5 sccm.

In certain embodiments, the functionalisation of the substrate comprises plasma polymerisation with the alkylamine at a power of 10 W or less and a flow rate of greater than 1 sccm.

Similar plasma polymer coatings may be obtained using alternate plasma reactor systems with a range of precursors as described herein.

One generalised method involves operating the plasma reactor under known conditions (precursor flowrate, pressure, RF power etc) and measuring the primary amine content of the resulting coating. If the measured primary amine content is lower than the desired range, it may be increased by decreasing the W/FM parameter (for example as described in Yasuda, Plasma Polymerization, Academic Press, New York, 1985), where W is the applied RF power, F is the precursor flowrate and M is the molecular weight of the precursor. This may be achieved by either decreasing the RE power, increasing the flowrate or a mixture of both. Alternatively, if the measured primary amine content is higher than the desired range the W/FM parameter should be increased.

In certain embodiments, the attaching of cells to the functionalised substrate comprises passive attachment of the cells to the substrate. For example, cells may be placed and/or cultured in the presence of the substrate and attachment of the cells obtained in this way. Other methods for attachment of the cells to the substrate are contemplated.

In certain embodiments, applying the product to the site to allow transfer of the cells from the product to the site is achieved by placing the product in direct contact with the site. For example, a wound healing product may be placed in direct contact with the wound.

In certain embodiments, applying the product to the site to allow transfer of the cells from the product to the site is achieved by indirect contact with the site, and allowing migration of the cells to the desired site. For example, a composition comprising the functionalised substrate and cells attached to the substrate may be administered to a subject and cells released from the product can move to a remote site of action. For example, a composition comprising particles could be delivered by implantation into a subject and cells transferred to a desired site of action by migration of the cells from the site of implantation to the desired site of action.

In certain embodiments, the method is used to deliver cells to a wound. In certain embodiments, the method is used to treat or heal a wound. Other applications are contemplated.

Certain embodiments of the present disclosure provide a method of delivering cells to a wound, the method comprising:
providing a wound healing product comprising an alkylamine functionalised substrate and cells for delivery to the wound attached to the functionalised substrate, wherein the alkylamine functionalised substrate comprises a surface density with an atomic ratio of primary amine to carbon of greater than 0.005; and
applying the product to the wound to allow transfer of the cells from the product to the wound;
thereby delivering cells from the wound healing product to the wound.

Certain embodiments of the present disclosure provide a product for delivery of cells to a site, as described herein.

Certain embodiments of the present disclosure provide a product for delivering cells to a site, the product comprising an alkylamine functionalised substrate and cells for delivery to the site attached to the functionalised substrate, wherein the alkylamine functionalised substrate comprises a surface density with an atomic ratio of primary amine to carbon of greater than 0.005.

In certain embodiments, the product comprises a bandage, a dressing, a gauze or a patch. In certain embodiments, the product comprises an implantable product. In certain embodiments, the product comprises a degradable product. In certain embodiments, the product comprises a composition. In certain embodiments, the product comprises particles or beads. Methods for producing a product comprising an alkylamine functionalised substrate are known in the art.

Cells are as described herein. In certain embodiments, the cells comprise multipotent cells. In certain embodiments, the cells comprise stem cells, such as adult stem cells. In certain embodiments, the cells comprise multipotent adult progenitor cells (MAPCs). In certain embodiments, the cells comprise multipotent stromal cells. In certain embodiments, the cells comprise multipotent stem cells capable of differentiating to form adipocytes, cartilage, bone, tendons, muscle, and skin. Other types of cells are contemplated.

In certain embodiments, the cells comprise mesenchymal stem cells. Methods for isolating cells, including MAPCs and mesenchymal stem cells, are known in the art.

Details of the site for delivery of cells are as described herein. In certain embodiments, the biological site comprises a site for tissue or cell repair, a site for tissue or cell production, a site for tissue or cell regeneration, a site benefiting from the delivery of cells, such cartilage, bone, fat, heart tissue and/or a site of neovascularisation. Other types of sites are contemplated.

Certain embodiments of the present disclosure provide a product for delivering cells to a biological site, the product comprising an alkylamine functionalised substrate and cells for delivery to the biological site attached to the functionalised substrate, wherein the alkylamine functionalised substrate comprises a surface density with an atomic ratio of primary amine to carbon of greater than 0.005.

In certain embodiments, the site comprises a wound. Examples of wounds are described herein and may include both open and closed wounds. In certain embodiments, the wound comprises an external wound. In certain embodiments, the wound comprises an open wound. In certain embodiments, the wound comprises a chronic wound. In certain embodiments, the wound comprises a chronic wound or an ulcer, such as a diabetic wound or a diabetic ulcer

Functionalisation of a substrate with an alkylamine is as described herein. In certain embodiments, the alkylamine functionalised substrate comprises a substrate functionalised with heptylamine and/or a substituted derivative thereof. In certain embodiments, the akylamine functionalised substrate comprises a heptylamine functionalised substrate. Other alkylamine functionalised substrates are as described herein.

Substrates are as described herein. In certain embodiments, the substrate comprises one or more polymers. Polymers are as described herein.

In certain embodiments, the substrate is a non-metal substrate.

In certain embodiments, the substrate comprises a silicone and/or a polyurethane. Other types of substrates are as described herein.

Characteristics of the surface density of the functionalised substrate are as described herein.

In certain embodiments, the surface density of the functionalised substrate comprises an atomic ratio of primary amine to carbon of greater than 0.006, greater than 0.007, greater than 0.008 or greater than 0.009.

In certain embodiments, the surface density of the functionalised substrate comprises an atomic ratio of primary amine to carbon of greater than 0.009.

In certain embodiments, the surface density comprises an atomic ratio of primary amine to carbon in the range from 0.005 to 0.04, 0.005 to 0.035, 0.005 to 0.03, 0.005 to 0.025, 0.005 to 0.02, 0.005 to 0.015, 0.005 to 0.01, 0.005 to 0.009, 0.005 to 0.008, 0.005 to 0.007, and 0.005 to 0.006.

In certain embodiments, the surface density comprises an atomic ratio of primary amine to carbon in the range from 0.009 to 0.04, 0.009 to 0.035, 0.009 to 0.03, 0.009 to 0.025, 0.009 to 0.02, 0.009 to 0.015 and 0.009 to 0.010.

In certain embodiments, the surface density comprises an atomic ratio of primary amine to carbon in the range from 0.009 to 0.04.

Methods for determining surface density are known in the art.

In certain embodiments, the surface density comprises an atomic ratio of primary amine to nitrogen of greater than one of 0.08, 0.09, 0.1, 0.11, 0.12, 0.13, 0.14, 0.15, 0.16, 0.17, 0.18 or 0.19.

In certain embodiments, the surface density comprises an atomic ratio of primary amine to nitrogen of greater than 0.08.

In certain embodiments, the surface density comprises an atomic ratio of primary amine to nitrogen in the range from° . 08 to 0.20, 0.08 to 0.19, 0.08 to 0.18, 0.08 to 0.17, 0.08 to 0.16, 0.08 to 0.15, 0.08 to 0.14, 0.08 to 0.13, 0.08 to 0.12, 0.08 to 0.11, 0.08 to 0.10, 0.08 to 0.09, 0.09 to 0.20, 0.09 to 0.19, 0.09 to 0.18, 0.09 to 0.17, 0.09 to 0.16, 0.09 to 0.15, 0.09 to 0.14, 0.09 to 0.13, 0.09 to 0.12, 0.09 to 0.11, 0.09 to 0.10, 0.10 to 0.20, 0.10 to 0.19, 0.10 to 0.18, 0.10 to 0.17, 0.10 to 0.16, 0.10 to 0.15, 0.10 to 0.14, 0.10 to 0.13, 0.10 to 0.12, 0.10 to 0.11, 0.11 to 0.20, 0.11 to 0.19, 0.11 to 0.18, 0.11 to 0.17, 0.11 to 0.16, 0.11 to 0.15, 0.11 to 0.14, 0.11 to 0.13, 0.11 to 0.12, 0.12 to 0.20, 0.12 to 0.19, 0.12 to 0.18, 0.12 to 0.17, 0.12 to 0.16, 0.12 to 0.15, 0.12 to 0.14, 0.12 to 0.13, 0.13 to 0.20, 0.13 to 0.19, 0.13 to 0.18, 0.13 to 0.17, 0.13 to 0.16, 0.13 to 0.15, 0.13 to 0.14, 0.14 to 0.20, 0.14 to 0.19, 0.14 to 0.18, 0.14 to 0.17, 0.14 to 0.16, 0.14 to 0.15, 0.15 to 0.20, 0.15 to 0.19, 0.15 to 18, 0.15 to 0.17, 0.15 to 0.16, 0.16 to 0.20, 0.16 to 0.19, 0.16 to 0.18, 0.16 to 0.17, 0.17 to 0.20, 0.17 to 0.19, 0.17 to 0.18, 0.18 to 0.20, 0.18 to 0.19, and 0.19 to 0.20.

In certain embodiments, the surface density comprises an atomic ratio of primary amine to nitrogen ratio in the range from 0.08 to 0.20.

Characteristics of the functionalisation of the substrate using plasma polymerisation are as described herein. In certain embodiments, the functionalisation of the substrate comprises plasma polymerisation with the alkylamine at a power of 20 W or less, 15 W or less, 10 W or less, or 2 W or less.

In certain embodiments, the functionalisation of the substrate comprises plasma polymerisation with the alkylamine at a power of 20 W or less. In certain embodiments, the functionalisation of the substrate comprises plasma polymerisation with the alkylamine at a power of 10 W or less.

In certain embodiments, the functionalisation of the substrate comprises plasma polymerisation with the alkylamine at a power in the range from 1 W to 10 W.

In certain embodiments, the functionalisation of the substrate comprises plasma polymerisation with the alkylamine at a flow rate of greater than 1 sccm. In certain embodiments, the functionalisation of the substrate comprises plasma polymerisation with the alkylamine at a flow rate of greater than 2 sccm. In certain embodiments, the functionalisation of the substrate comprises plasma polymerisation with the alkylamine at a flow rate of greater than 3 sccm. In certain embodiments, the functionalisation of the substrate comprises plasma polymerisation with the alkylamine at a flow rate of greater than 4 sccm. In certain embodiments, the functionalisation of the substrate comprises plasma polymerisation with the alkylamine at a flow rate of greater than 5 sccm.

In certain embodiments, the functionalisation of the substrate comprises plasma polymerisation with the alkylamine at a flow rate in the range of 1 to 10 sccm. In certain embodiments, the functionalisation of the substrate comprises plasma polymerisation with the alkylamine at a flow rate in the range of 1 to 5 sccm.

In certain embodiments, the functionalisation of the substrate comprises plasma polymerisation with the alkylamine at a power of 10 W or less and a flow rate of greater than 1 sccm.

In certain embodiments, the attaching of cells to the functionalised substrate comprises passive attachment of the cells to the substrate. For example, cells may be placed and/or cultured in the presence of the substrate and attachment of the cells obtained in this way. Other methods for attachment of the cells to the substrate are contemplated. The number of cells may be selected to meet the desired use.

In certain embodiments, applying the product to the site to allow transfer of the cells from the product to the site is achieved by placing the product in direct contact with the site. For example, a wound healing product may be placed in directed contact with a wound.

In certain embodiments, applying the product to allow transfer of the cells from the product to the site is achieved by indirect contact with the site, and allowing migration of the cells to the desired site. For example, a composition comprising the functionalised substrate and cells attached to the substrate may be administered to a subject and cells released from the product can move to a remote site of action. For example, a composition comprising particles could be delivered by implantation into a subject and cells transferred to a desired site of action by migration of the cells from the site of implantation to the desired site of action.

A suitable number of cells may be attached to the substrate.

In certain embodiments, at least 1×10⁴ cells attached to the substrate are provided.

In certain embodiments, at least 1×10⁴, at least 2×10⁴ cells, at least 4×10⁴ cells, at least 1×10⁵ cells, or at least 2×10⁵ cells attached to the substrate are provided.

In certain embodiments, 1×10⁴ to 2×10⁵ cells, 2×10⁴ to 2×10⁵ cell, 4×10⁴ to 2×10⁵ cells, 8×10⁴ to 2×10⁵ cells, 1×10⁵ to 2×10⁵ cells, 1×10⁴ to 1×10⁵ cells, 2×10⁴ to 1×10⁵ cells, 4×10⁴ to 1×10⁵ cells, 8×10⁴ to 1×10⁵ cells, 1×10⁴ to 8×10⁴ cells, 2×10⁴ to 8×10⁴ cells, 4×10⁴ to 8×10⁴ cells, 1×10⁴ to 4×10⁴ cells, 2×10⁴ to 4×10⁴ cells, or 1×10⁴ to 2×10⁴ cells attached to the substrate are provided.

In certain embodiments, the substrate comprises cells at a density on the substrate of at least 1×10⁴ cells per cm², at least 1.2×10⁴ cells per cm², at least 2.5×10⁴ cells per cm², at least 5×10⁴ cells per cm², at least 1×10⁵ cells per cm², at least 1.2×10⁵ cells per cm², at least 2×10⁵ cells per cm², or at least 2.5×10⁵ cells per cm².

In certain embodiments, the product is used to deliver cells to a wound. In certain embodiments, the product is used to treat or heal a wound. Other applications are contemplated.

Certain embodiments of the present disclosure provide a wound healing product comprising an alkylamine functionalised substrate and cells for healing a wound attached to the functionalised substrate, wherein the the alkylamine functionalised substrate comprises a surface density with an atomic ratio of primary amine to carbon of greater than 0.005.

Certain embodiments of the present disclosure provide a wound healing product comprising:
an alkylamine functionalised substrate, wherein the alkylamine functionalised substrate comprises a surface density with an atomic ratio of primary amine to carbon of greater than 0.005; and
cells for healing a wound attached to the substrate.

In certain embodiments, the product comprises a composition.

Certain embodiments of the present disclosure provide a composition comprising an alkylamine functionalised substrate and cells for healing a wound attached to the substrate, wherein the alkylamine functionalised substrate comprises a surface density with an atomic ratio of primary amine to carbon of greater than 0.005.

In certain embodiments, composition comprises a wound healing composition.

In certain embodiments, the composition is suitable for topical application, topical administration or topical delivery to a subject. Topical formulations and topical products are as described herein. Other forms of delivery of cells are contemplated.

In certain embodiments, the composition is suitable for topical application, topical administration or topical delivery to a wound.

The dose and frequency of topical administration may be determined by one of skill in the art.

Examples of forms for topical administration include delivery by way of a gel, an ointment, a cream, a lotion, a foam, an emulsion, a suspension, a spray, an aerosol, a solution, a liquid, a powder, a semi-solid, a gel, a jelly, a suppository; a solid, an ointment, a paste, a tincture, a linament, a patch, or release from a patch, a bandage, gauze or dressing. Other forms of topical delivery are contemplated.

In certain embodiments, the form of administration comprises a patch, a bandage, a gauze, or a dressing.

Methods for incorporating substrates into products for topical release are known in the art, for example as described in Boateng J. S. et al (2008) “Wound healing dressings and drug delivery systems: a review” J. Pharm Sci. 97(8): 2892-2923 and “Delivery System Handbook for Personal Care and Cosmetic Products: Technology” (2005) by Meyer Rosen, published William Andrew Inc, Norwich New York.

In certain embodiments, the composition is suitable for delivery to a subject by one or more of intravenous administration, by aerosolized administration, by parenteral administration, by implant, by subcutaneous injection, intraarticularly, rectally, intranasally, intraocularly, vaginally, or transdermally.

In certain embodiments, the composition comprises other compounds that enhance, stabilise or maintain the activity of the cells for delivery and/or their delivery or transfer.

In certain embodiments, it may be desirable to administer the composition parenterally (such as directly into the joint space) or intraperitoneally. For example, solutions or suspensions can be prepared in water suitably mixed with a surfactant such as hydroxy-propylcellulose. Dispersions can also be prepared in glycerol, liquid polyethylene glycols and mixtures thereof in oils.

In certain embodiments, it may be desirable to administer the composition by injection. Forms suitable for injectable use include sterile aqueous solutions or dispersions and sterile powders for the extemporaneous preparation of sterile injectable solutions or dispersions. A carrier can be a solvent or dispersion medium containing, for example, water, ethanol, polyol (e.g., glycerol, propylene glycol and liquid polyethylene glycol), suitable mixtures thereof, and vegetable oils.

In certain embodiments, it may be desirable to administer the composition intravenously. Compositions containing the composition described herein suitable for intravenous administration may be formulated by a skilled person.

Certain embodiments of the present disclosure provide a method of preventing or treating a subject with a disease, condition or state that would benefit from the delivery of suitable cells to the subject. Methods for delivery of cells to a subject are as described herein.

In certain embodiments, the subject is a human or animal subject. In certain embodiments, the subject is a human subject.

In certain embodiments, the subject is suffering from diabetes.

In certain embodiments, the subject is a mammalian subject, a livestock animal (such as a horse, a cow, a sheep, a goat, a pig), a domestic animal (such as a dog or a cat) and other types of animals such as monkeys, rabbits, mice, laboratory animals, birds and fish. Other types of animals are contemplated. Veterinary applications of the present disclosure are contemplated.

In certain embodiments, the subject is suffering from a wound. In certain embodiments, the subject is suffering from an open wound. In certain embodiments, the subject is suffering from a chronic wound. In certain embodiments, the subject is susceptible to developing a chronic wound or an ulcer. In certain embodiments, the subject is suffering from a diabetic wound or a diabetic ulcer.

Certain embodiments of the present disclosure provide a method of treating or healing a wound in a subject.

Certain embodiments of the present disclosure provide a method of healing or treating a wound, the method comprising delivering cells to the wound using a product or a composition as described herein.

Certain embodiments of the present disclosure provide a method of treating a wound, the method comprising applying to the wound a product comprising an alkylamine functionalised substrate and cells for healing the wound attached to the functionalised substrate, wherein the alkylamine functionalised substrate comprises a surface density with an atomic ratio of primary amine to carbon of greater than 0.005.

The delivery of cells utilises a therapeutically effective amount of a product as described herein.

The term “therapeutically effective amount” refers to that amount which is sufficient to effect prevention and/or treatment, when administered to a subject. The dose and frequency of administration may be determined by one of skill in the art.

In certain embodiments, the method comprises providing at least 1×10⁴ cells, at least 2×10⁴ cells, at least 4×10⁴ cells, at least 1×10⁵ cells, or at least 2×10⁵ cells attached to the substrate.

In certain embodiments, the method comprises providing 1×10⁴ to 2×10⁵ cells, 2×10⁴ to 2×10⁵ cells, 4×10⁴ to 2×10⁵ cells, 8×10⁴ to 2×10⁵ cells, 1×10⁵ to 2×10⁵ cells, 1×10⁴ to 1×10⁵ cells, 2×10⁴ to 1×10⁵ cells, 4×10⁴ to 1×10⁵ cells, 8×10⁴ to 1×10⁵ cells, 1×10⁴ to 8×10⁴ cells, 2×10⁴ to 8×10⁴ cells, 4×10⁴ to 8×10⁴ cells, 1×10⁴ to 4×10⁴ cells, 2×10⁴ to 4×10⁴ cells, or 1×10⁴ to 2×10⁴ cells attached to the substrate.

In certain embodiments, the method comprises providing cells at a density on the substrate of at least 1×10⁴ cells per cm², at least 1.2×10⁴ cells per cm², at least 2.5×10⁴ cells per cm², at least 5×10⁴ cells per cm², at least 1×10⁵ cells per cm², at least 1.2×10⁵ cells per cm², at least 2×10⁵ cells per cm², or at least 2.5×10⁵ cells per cm².

The term “prevent”, and related terms such as “prevention” and “preventing”, refer to obtaining a desired effect in terms of arresting or suppressing the appearance of one or more symptoms in the subject.

The term “treat”, and related terms such as “treating” and “treatment”, refer to obtaining a desired effect in terms of improving the condition of the subject, ameliorating, arresting, suppressing, relieving and/or slowing the progression of one or more symptoms in the subject, a partial or complete stabilisation of the subject, a regression of the one or more symptoms, or a cure of a disease, condition or state in the subject.

Certain embodiments of the present disclosure provide a method of producing a wound healing product, as described herein.

Certain embodiments of the present disclosure provide a method of producing a wound healing product comprising cells for healing a wound attached to a substrate, the method comprising attaching the cells to the substrate which has been functionalised with an alkylamine and comprises a surface density with an atomic ratio of primary amine to carbon of greater than 0.005.

In certain embodiments, the method comprises attaching at least 1×10⁴ cells, at least 2×10⁴ cells, at least 4×10⁴ cells, at least 1×10⁵ cells, or at least 2×10⁵ to the substrate.

In certain embodiments, the method comprises attaching 1×10⁴ to 2×10⁵ cells, 2×10⁴ to 2×10⁵ cell, 4×10⁴ to 2×10⁵ cells, 8×10⁴ to 2×10⁵ cells, 1×10⁵ to 2×10⁵ cells, 1×10⁴ to 1×10⁵ cells, 2×10⁴ to 1×10⁵ cells, 4×10⁴ to 1×10⁵ cells, 8×10⁴ to 1×10⁵ cells, 1×10⁴ to 8×10⁴ cells, 2×10⁴ to 8×10⁴ cells, 4×10⁴ to 8×10⁴ cells, 1×10⁴ to 4×10⁴ cells, 2×10⁴ to 4×10⁴ cells, or 1×10⁴ to 2×10⁴ cells to the substrate.

In certain embodiments, the method comprises attaching cells at a density to the substrate of at least 1×10⁴ cells per cm², at least 1.2×10⁴ cells per cm², at least 2.5×10⁴ cells per cm², at least 5×10⁴ cells per cm², at least 1×10⁵ cells per cm², at least 1.2×10⁵ cells per cm², at least 2×10⁵ cells per cm², or at least 2.5×10⁵ cells per cm².

Certain embodiments of the present disclosure provide a method of producing a wound healing product comprising cells for healing a wound attached to a substrate, the method comprising:
functionalising the substrate with a plasma polymerised alkylamine, wherein the functionalising of the substrate produces a substrate with a surface density with an atomic ratio of primary amine to carbon of greater than 0.005; and attaching the cells for healing a wound to the functionalised substrate.

Certain embodiments of the present disclosure provide a wound healing product produced by a method as described herein.

Certain embodiments of the present disclosure provide a method of modifying a substrate for attachment of cells, as described herein.

Certain embodiments of the present disclosure provide a method of modifying a substrate for attachment of cells, the method comprising exposing the substrate to plasma polymerisation with an alkylamine to modify the substrate, wherein the plasma polymerization with the alkylamine produces a substrate with a surface density with an atomic ratio of primary amine to carbon of greater than 0.005.

Certain embodiments of the present disclosure provide a substrate modified by a method as described herein. Certain embodiments of the present disclosure provide a wound healing product comprising a substrate modified by a method as described herein.

Certain embodiments of the present disclosure provide a method of functionalising a substrate for attachment of cells, as described herein.

Certain embodiments of the present disclosure provide a method of functionalising a substrate for attachment of cells, the method comprising modifying the substrate by plasma polymerisation with an alkylamine to functionalise the substrate, wherein the plasma polymerization with the alkylamine produces a surface density with an atomic ratio of primary amine to carbon of greater than 0.005.

Certain embodiments of the present disclosure provide a substrate functionalised by a method as described herein.

Certain embodiments of the present disclosure provide a wound healing product comprising a substrate functionalised by a method as described herein.

Certain embodiments of the present disclosure provide an alkylamine functionalised substrate, wherein the substrate comprises a surface density of primary amine to carbon ratio of greater than 0.005.

In certain embodiments, the substrate comprises a polymer.

In certain embodiments, the substrate is a non-metal substrate.

Certain embodiments of the present disclosure provide a wound healing product comprising an alkylamine functionalised substrate and cells for healing a wound attached to the functionalised substrate, wherein the alkylamine functionalised substrate comprises a surface density with an atomic ratio of primary amine to carbon of greater than 0.005.

Standard techniques may be used for cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), herein incorporated by reference.

The present disclosure is further described by the following examples. It is to be understood that the following description is for the purpose of describing particular embodiments only and is not intended to be limiting with respect to the above description.

### EXAMPLE 1—CULTURING OF MAPCs

MAPCs were cultured as described in Reading, James L., Jennie HM Yang, Shereen Sabbah, Ania Skowcra, Robin R. Knight, Jcf Pinxteren, Bart Vacs et al. “Clinical-grade multipotent adult progenitor cells durably control pathogenic T cell responses in human models of transplantation and autoimmunity.” The Journal of Immunology 190, no. 9 (2013): 4542-4552.

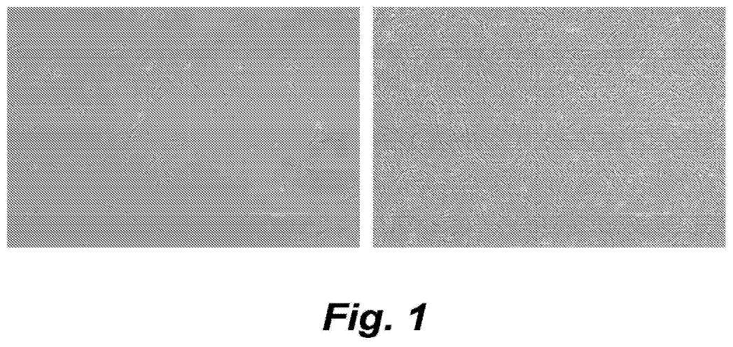

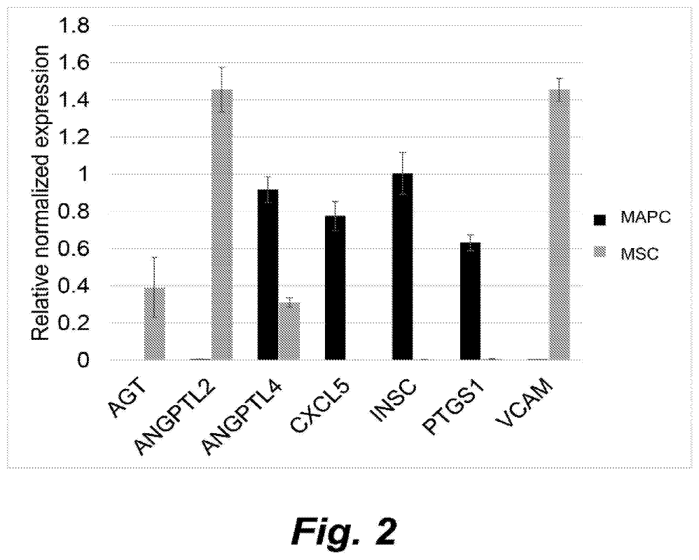

The cells exhibited appropriate growth rates and morphologies, as shown in FIG. 1. Analysis of mRNA expression through RNA isolation, cDNA synthesis and qPCR expression was carried out. It was found that the expression levels of the MAPCs compared with donor-matched MSCs fell within pre-defined tolerances, as shown in FIG. 2.

### EXAMPLE 2—Surface substrate selection and functionalisation

(i) Surface Substrate Selection

Initial studies were carried out using medical grade silicone sheets.

(ii) Treating Surfaces using Plasma Polymerisation

A set of initial monomers was chosen (shown in Table 1) for analysis. The monomers were selected to allow analysis of different monomers for functionalisation of the substrate and to allow a comparison of (i) saturated versus unsaturated monomers; and (ii) acid monomers versus amine monomers.

TABLE 1
Monomers selected for screening.

| | | | |
|---|---|---|---|
| Acrylic acid | Acid | Unsaturated | 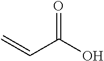 |
| Propanoic acid | Acid | Saturated | 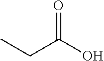 |
| Allylamine | Amine | Unsaturated | 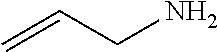 |
| Heptylamine | Amine | Saturated | 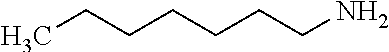 |

All samples were degassed and treated with the plasma for 20 minutes substantially according to the following protocol: Michelmore, Andrew, Petra Gross-Kosehe, Sameer A. Al-Bataineh, Jason D. Whittle, and Robert D. Short. “On the effect of monomer chemistry on growth mechanisms of nonfouling PEG-like plasma polymers. “Langmuir”29, no. 8 (2013): 2595-2601.

Reagents & materials:
Heptylamine (Aldrich 126802-10OG)
Liquid nitrogen
A parallel plate RF (13.56 MHz) plasma reactor consisting of a 0.25 m steel cylinder with an internal diameter of 0.3 m.

A simplified diagram of the system used is shown below:

Monomer was de-gassed by repeated freeze-thaw cycling using liquid nitrogen and samples placed into the reactor to de-gas. When the chamber was below 5×10⁴ mbar, the samples were appropriately de-gassed. Pressure noted.

The monomer flow rate was adjusted to the desired level and ensured it was stable. RF power was applied and the plasma colour and intensity ensured to be within appropriate thresholds. Run for 20 minutes.

When the plasma cycle was completed, the running pressure was noted, turned off the RF power and allowed the monomer to flow for an additional 10 minutes.

The monomer flow valve was turned off and samples pumped back down to base pressure. The chamber was vented and the samples removed and stored in sealed dry containers at room temperature.

For the optimised heptylamine coating, the following parameters were required:
Monomer: Heptylamine
Base pressure: 1×10⁻⁴
Flow rate of heptylamine: 2-4 sccm
RF power: 5 W

Samples were stored in sealed containers at room temperature prior to use. Cell assays were carried out within 4 weeks of surface coating.

(iii) Transfer Assays (in vitro)

The transfer assay as described below was an in vitro model used to assess the transfer of cells from a surface into a model would site. The model wound site is human de-epidermised dermis.

After the polymer patch had been in place for 24 hours, the patch was removed and the metabolic conversion of MTT into an insoluble formazan product was used to determine the location and viability of cells; whether they were on the polymer still, or whether they had they migrated to the dermis.

The purpose of these studies was to devise a substrate that can support healthy maintenance of the MAPCs, but also allows the cells to migrate to a wound site.

The transfer assays were carried out according to the following protocol:

### Abbreviations

DED De-Epidermised Dermis
BSC Biological Safety Cabinet
PBS Phosphate buffered saline
HCl Hydrochloric acid
MTT 3-(4,5-dimethylthiazol-2-yl)-2,5-diphenyltetrazolium bromide
MAPC Multipotent Adult Progenitor Cell

### Reagents & Materials

De-epidermised dermis
MTT reagent (Invitrogen M6469 1 g)
Isopropanol
Hydrochloric acid 1M.
Phosphate buffered saline (PBS)

Day 1—Seeding MAPCs onto patches

Candidate patches (12×12 mm) were placed into wells of a 6-well plate and sterilised under UV within a Class II BSC for 20 minutes.

Cell seeding rings with an internal area of 0.79cm² were placed onto the patches and added 200 μl of cell suspension into the cell seeding ring (20×10³ cells). A suspension of 100×10³ MAPCs/m1 was used.

A control plate of MAPCs was prepared on tissue culture plastic. 240×10³ MAPCs were placed into fibronectin-treated wells of a 6-well plate in triplicate. This cell density was the same as the patches per unit area.

Incubated under hypoxic conditions for 24 hours.

Day 2—Transfer of patches onto DED

The cell seeding rings were removed and the patch was placed onto cut pieces of DED in a 6-well plate. It was ensured that the patches were face down with the cells in contact with the papillary surface of the DED. When placing the patch onto the DED, a rolling motion was used from one corner without dragging the patch. A cell culture grid was placed onto the patch to weight it down and added enough media to cover (3 ml).

Incubated for 24 hours.

Day 3—Assessment of MAPC metabolic activity with MTT

MTT solution was prepared (0.5 mg/ml in PBS).

Polymer patches were removed from the DED and placed into corresponding 6-well plates, seeded side upwards then 3 ml of MTT solution was added into each well. Incubated at 37° C. for 2-4 hours, checking regularly. Once appropriate colour development had occurred, the MTT solution was aspirated and the wells were imaged.

Acidified isopropanol (0.04 N) was prepared by adding 8 ml of 1M HCl to 200 ml isopropanol.

A semi-quantification of the insoluble formazan product was carried out by solubilising the product with acidified isopropanol. 3 ml of acidified isopropanol was added to each well. The 6-well plates were placed onto a shaker until the colour eluted. This solution was transferred into 96-well plates (200 μl/well) in triplicate and the absorbance measured at 570 nm. Appropriate negative controls were included and the positive control was diluted appropriately 1 in 12.

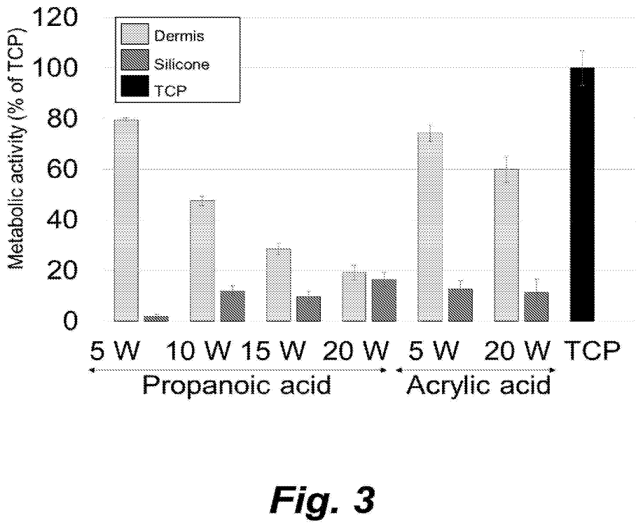

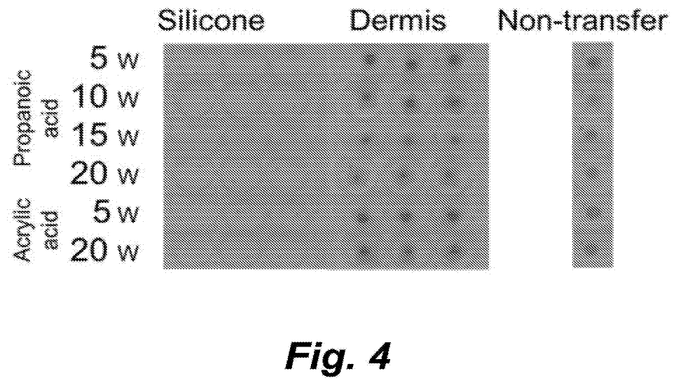

An initial set of screening was carried out with a fixed monomer flow rate of 4±0.5 sccm. Low power acid-based surfaces were able to deliver metabolically active MAPCs into a model wound bed with a metabolic activity approximately 80% that of fibronectin coated TCP (tissue culture plastic, the current standard for MAPC cell culture (FIGS. 3 and 4).

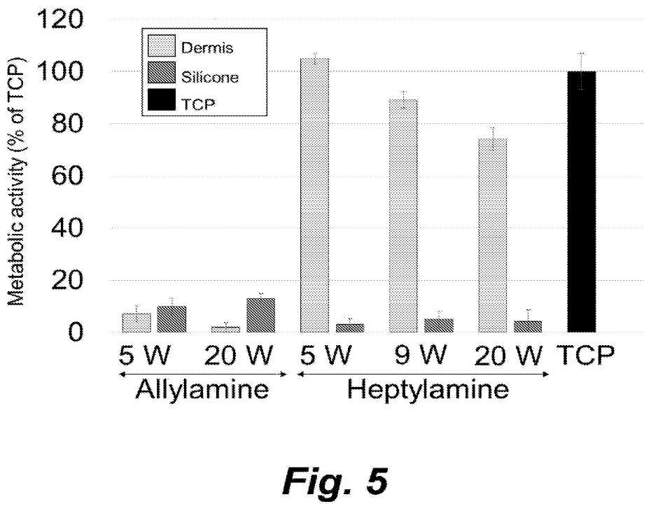

Referring to FIG. 5, allylamine performed less well on initial tests whereas heptylamine was found to be a superior surface coating for the delivery of MAPCs, indicating that a saturated amine monomer produced a coated surface which was superior to that coated with an unsaturated amine monomer.

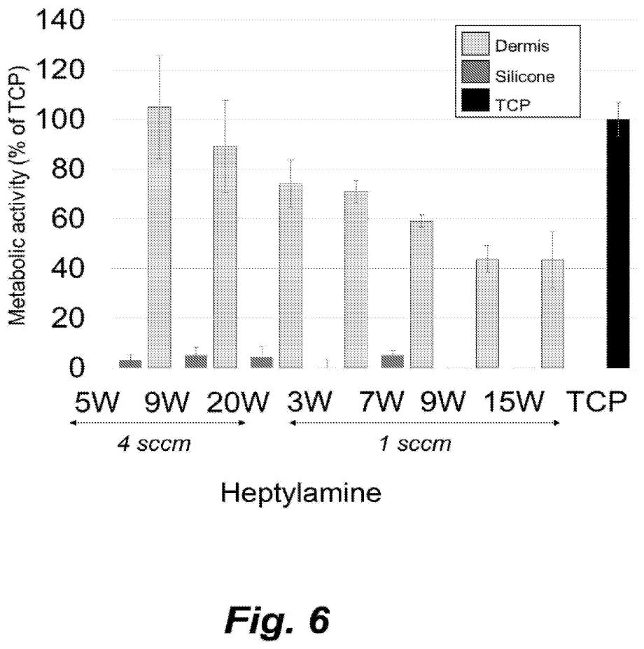

Upon further investigation it was also shown that, of the selected conditions, the higher flow rate (4 sccm) and lower power (5 W) were the most effective conditions in creating a patch to deliver MAPCs (FIG. 6).

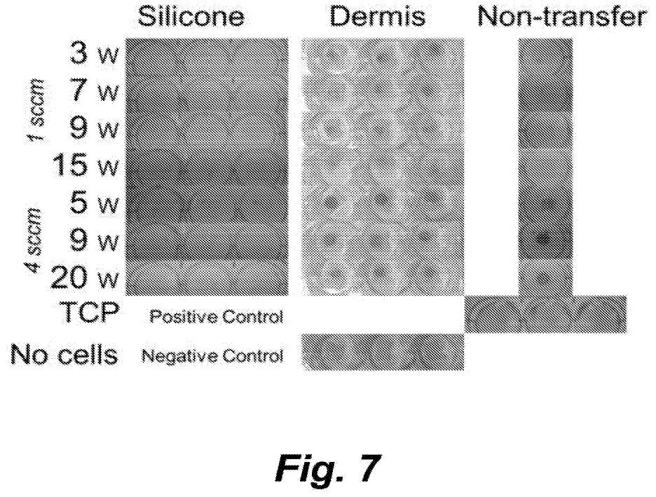

FIG. 7 shows transfer assay in vitro with metabolic activity quantified using MTT reagent. Lower power levels and higher flow rates were more favourable for cell transfer. Patches with a 5 W, 4sccm heptylamine plasma polymerisation were able to deliver cells to the dermis with a metabolic activity approximately 100% that of fibronectin coated TCP. FIG. 7 shows images of MTT stained silicone and dermis. Purple colour indicates metabolising cells.

### EXAMPLE 3—PHENOTYPE OF MAPCs ON PATCH

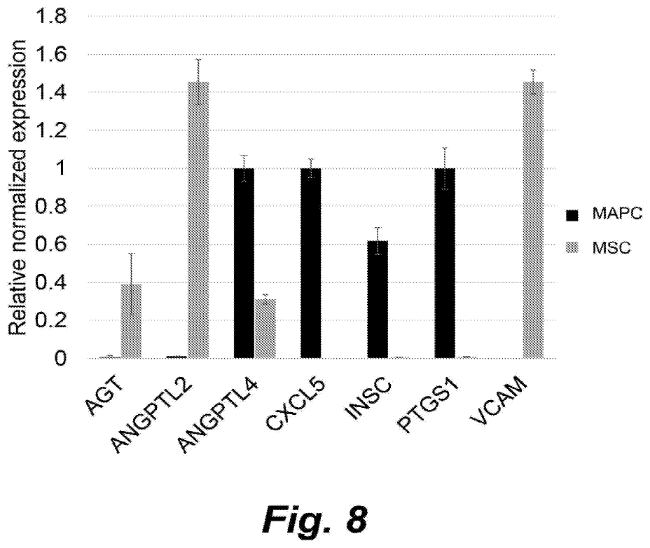

As described above, the silicone substrate using a plasma polymer from Heptylamine (5 W) was shown to be the best candidate for the delivery of MAPCs. It was essential to show that the MAPCs remain as MAPCs on this novel surface. MAPCs were cultured on the candidate patch for 48 hours, collected and analysis of mRNA expression through RNA isolation, cDNA synthesis and qPCR expression using the procedure as described above. The results show that the MAPCs remained within defined tolerances (FIG. 8), indicating that at the point of delivery from the patch, the cells remain within therapeutic tolerances.

### EXAMPLE 4—ANALYSIS OF SURFACES USING XPS

X-ray photoelectron spectroscopy (XPS) was used to characterise the surfaces.

The technique delivers relative atomic ratios and through fitting of the C1s peak, different carbon-based functional groups can be determined.

XPS-X-ray photoelectron spectroscopy is as described in Ruiz, Juan-Carlos, Shima Taheri, Andrew Michelmore, David E. Robinson, Robert D. Short, Krasimir Vasilev, and Renate Förch. “Approaches to Quantify Amine Groups in the Presence of Hydroxyl Functional Groups in Plasma Polymerized Thin Films.” Plasma Processes and Polymers (2014) and Beamson, Graham, and David Briggs. “High resolution XPS of organic polymers.” (1992).

Reagents & materials: SPECS SAGE XPS system with Phoibos 150 hemispherical analyser with a 900 take-off angle and 9-channel detector.

The spectra were imported into Casa XPS software. The spectra were charge corrected relative to the aliphatic Cls carbon peak at 285 eV and a linear background was used. The default line shape of GL30 (30% Lorentzian, 70% Gaussian) was used. The regions detailed below were applied.

The created regions within the survey corresponding with the elements of interest are shown in Tables 2 to 4.

TABLE 2

| | Approximate Binding Energy | Relative Sensitivity Factor |
|---|---|---|
| Shell | (eV) | (R.S.F) |
| O1s | 532 | 2.85 |
| N1s | 399 | 1.77 |
| C1s | 285 | 1 |
| Si2p | 99 | 0.865 |

TABLE 3
ACIDS (C-Peak integration)
Table 3. Values derived from literature from Beamson et al (1992).

| | | Position | Position | |
|---|---|---|---|---|
| Label | Bonds | (eV) | (Relative to CH) | Area |
| C1 | CH | 285 | — | — |
| C2 | COOH | 289.2 | +4.2 | — |
| C3 | bCOOH | 285.7 | +0.7 | COOH x |
| | | | | 1 |
| C4 | C═O | 287.9 | +2.9 | — |
| C5 | C—OH | 286.55 | 1.55 | — |

TABLE 4
AMINES (C-Peak integration)
Table 4. Values derived from literature Riuz et al (2014).

| | | Position | Position |
|---|---|---|---|
| Label | Bonds | (eV) | (relative to C1) |
| C1 | C—C, C═C | 285 | — |
| C2 | C—NHₓ, C—C═N | 286.1 | +1.1 |
| C3 | C═N, C≡N, C—O | 286.8 | +1.8 |
| C4 | C═O, O=C—N | 287.9 | +2.9 |
| C5 | COOR | 289.2 | +4.2 |

Survey scans were used to calculate the relative atomic ratios and the carbon peaks were integrated to determine possible ratios of functional groups using relative shifts detailed below (Table 3 & Table 4).

TABLE 5
Values used to calculate the C-peak integrations
of acid based plasma polymers.

| | | Position |
|---|---|---|
| Label | Bonds | (eV) |
| C1 | CH | 285 |
| C2 | COOH | 289 |
| C3 | bCOOH | 285.7 |
| C4 | C═O | 287.9 |
| C5 | C—OH | 286.55 |

TABLE 6
Values used to calculate the C-peak integrations
of amine based plasma polymers.

| | | Position |
|---|---|---|
| Label | Bonds | (eV) |
| C1 | C—C, C═C | 285 |
| C2 | C—NHₓ, | 286.1 |
| C3 | C═N, C≡N, | 286.8 |
| | C—O | |
| C4 | C═O, O═C—N | 288.2 |
| C5 | COOR | 289.2 |

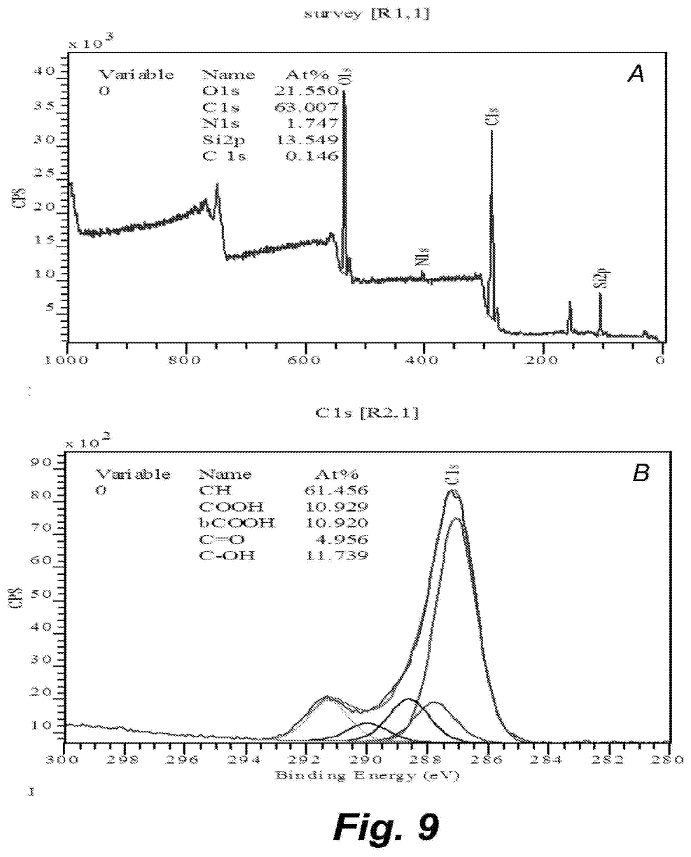

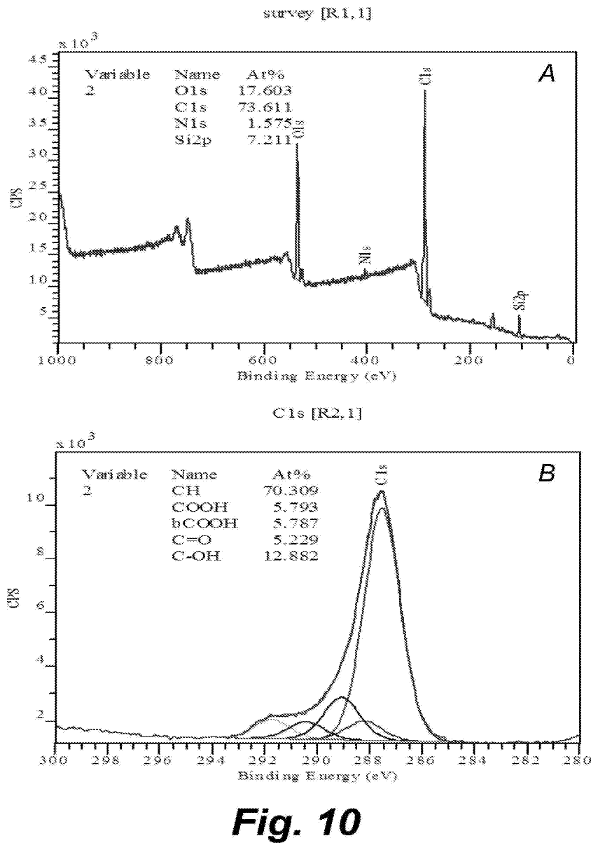

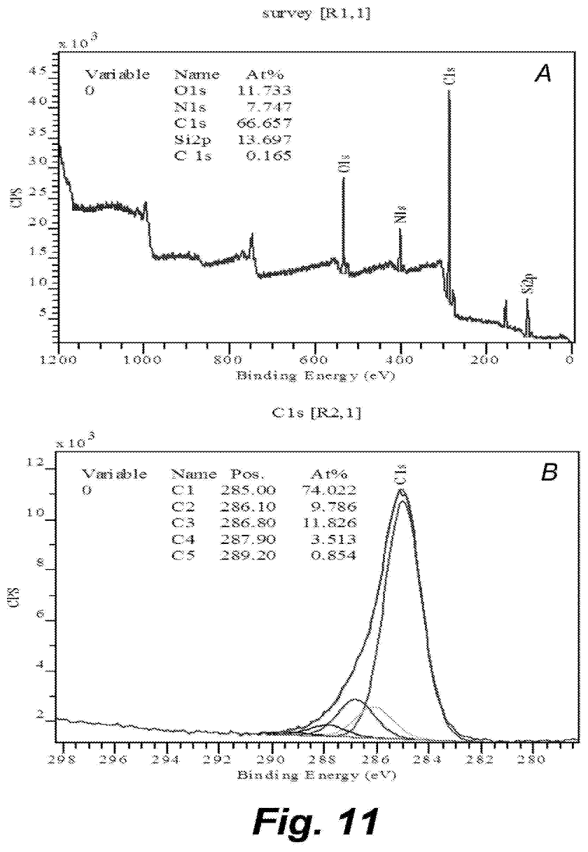

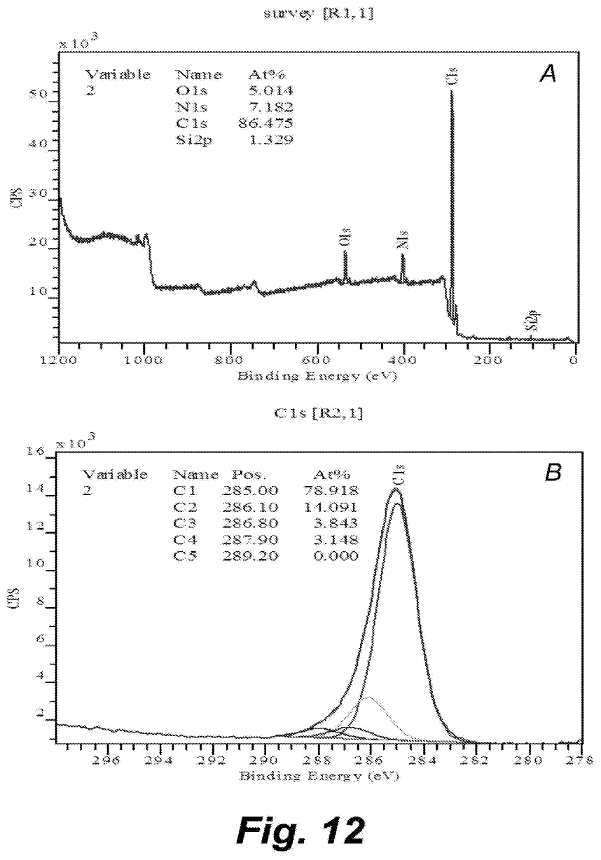

Typical scans for the candidate monomers are shown in FIG. 9 to FIG. 12.

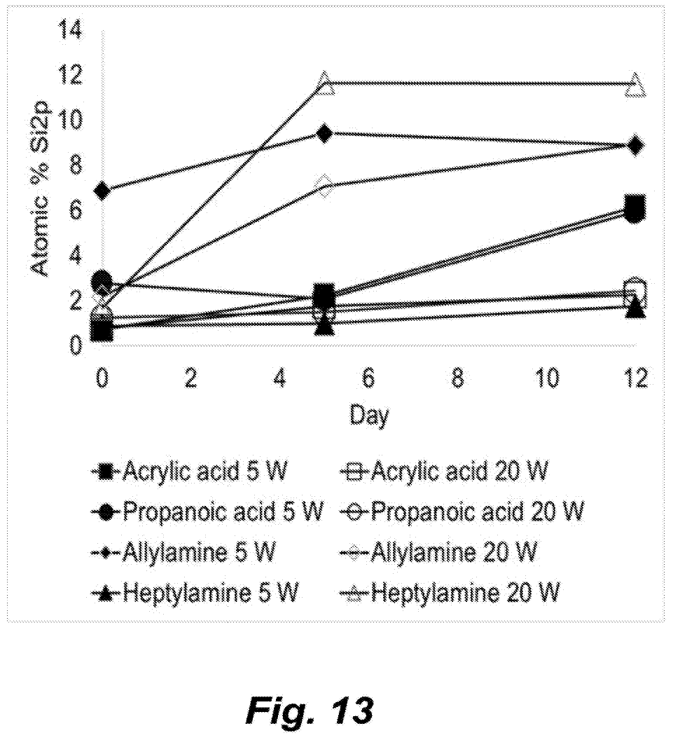

The mobility of the silicone surface substrate caused the levels of oxygen and silicon to be higher than expected and these levels increased over time. This effect was greater in low power surface coatings but evident in all surfaces (FIG. 13).

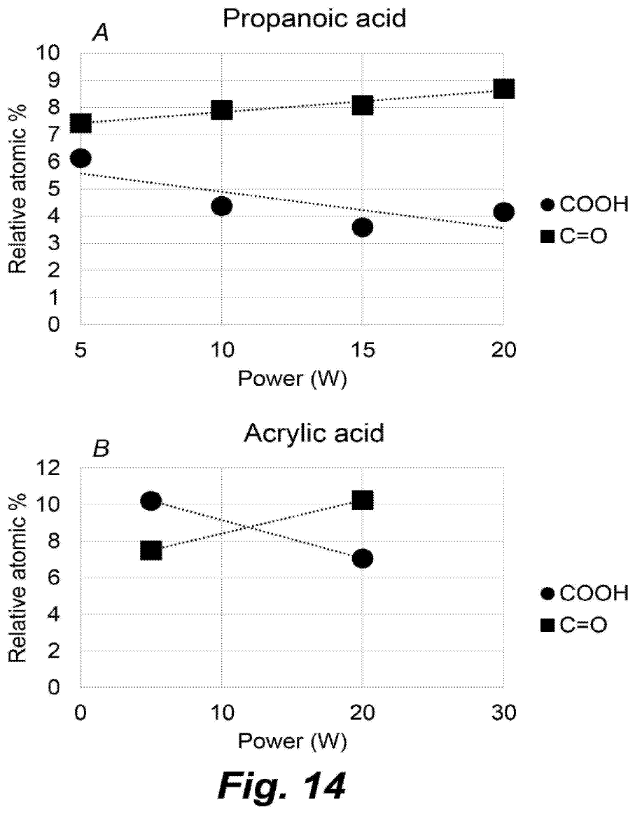

The functional groups in the acid based plasma surfaces can be accurately and reliably determined using C-peak integration and when we compare the XPS data from the batches used in transfer assays to the cell responses, trends can be seen. FIG. 4 shows that an increase in RF power corresponds to a decrease in cell transfer. This corresponds to linear trends in the acid functionality. As power increases, there is a decrease in cell transfer and in COOH groups. This is seen with a corresponding increase in C═0 groups as seen for both propanoic and acrylic acids in FIG. 14.

It was found that amine modified surfaces were more difficult to quantify. Although both power and flow rate had a clear effect on the effectiveness of patches for MAPC transfer, these differences could not be readily correlated with the elemental analysis of the XPS results. That being said, it was found that the heptylamine N/C ratios were a consistent 12-13%, although neither the carbon nor the nitrogen peaks were distinct enough to reveal data regarding differences in functionality.

### EXAMPLE 5—OPTIMISATION OF MAPC TRANSFER USING PRIMARY AMINE QUANTIFICATION

The following outlines data obtained through surface characterisation of amine-based surface coatings. Up until this point, the patches have been defined by their fabrication variables. Here the aim was to define them by the surface density of primary amines, and to determine the optimum value for MAPC transfer using an in vitro skin model.

### Methods

Heptylamine plasma polymers were deposited onto silicone substrates as described above. The flow rate was calculated as 1 sccm and powers of 2, 5, 8, 10, 15 & 20 W were used for a total of 6 batches.

An in vitro transfer assay was performed to validate all patches based on the method described above. This identifies the location of viable cells.

A method to tag primary amines using 4-(trifluoromethyl)benzaldehyde (TFBA) was used and is described by Ruiz JC, Taheri S, Michelmore A, Robinson DE, Short RD, Vasilev K, et al. Approaches to Quantify Amine Groups in the Presence of Hydroxyl Functional Groups in Plasma Polymerized Thin Films. Plasma Processes and Polymers 2014;11:888-96. Briefly, the patches were exposed to TFBA vapour (at 45° C.) for 3 hours then scanned using the XPS. The Quantitative Elemental Analysis (QEA) method was then used to derive the ratios of primary amine.

### Results & Discussion

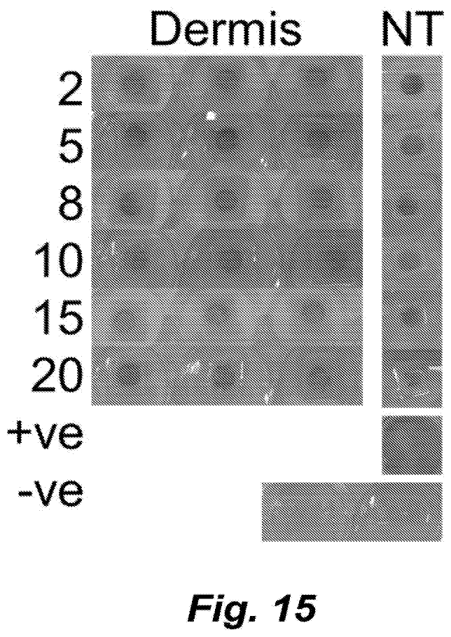

The transfer assay performed as previously demonstrated; as power increased above 5 W, there was a decrease in the number of viable cells transferred. FIG. 15 shows the images of dermis and silicone patches.

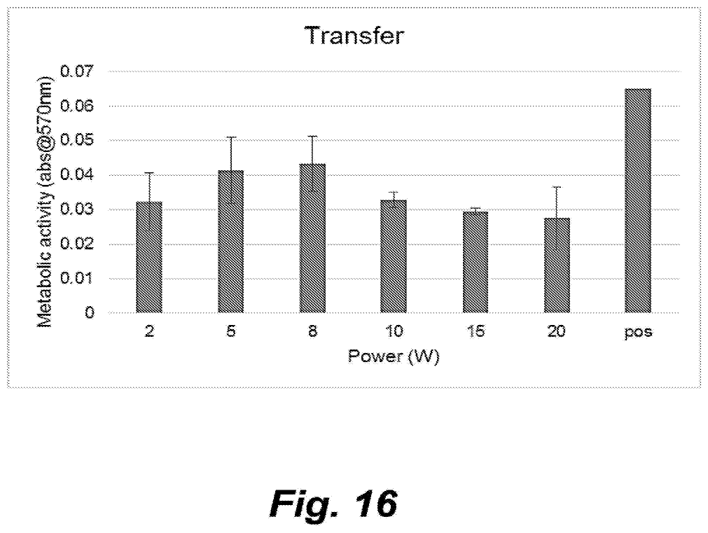

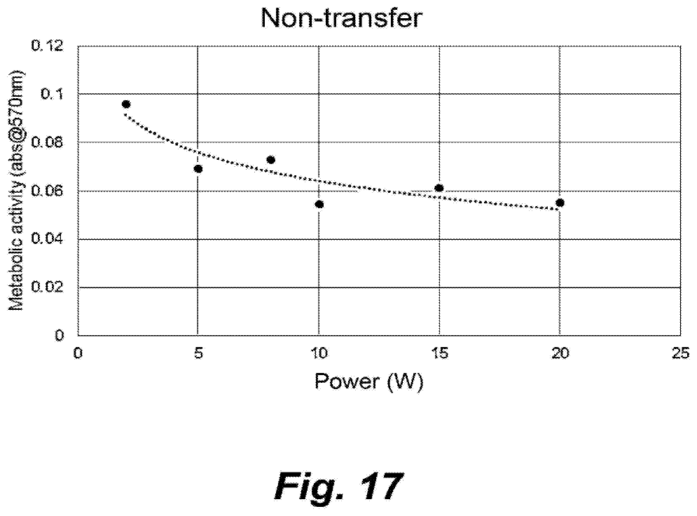

In this assay, for transfer purposes 5 & 8 W appeared optimum (FIG. 16). For culture alone, the lower power patch (2 W) appeared optimum (FIG. 17).

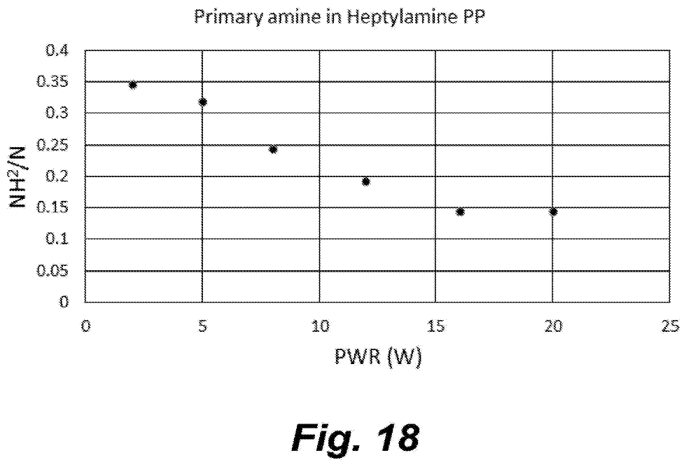

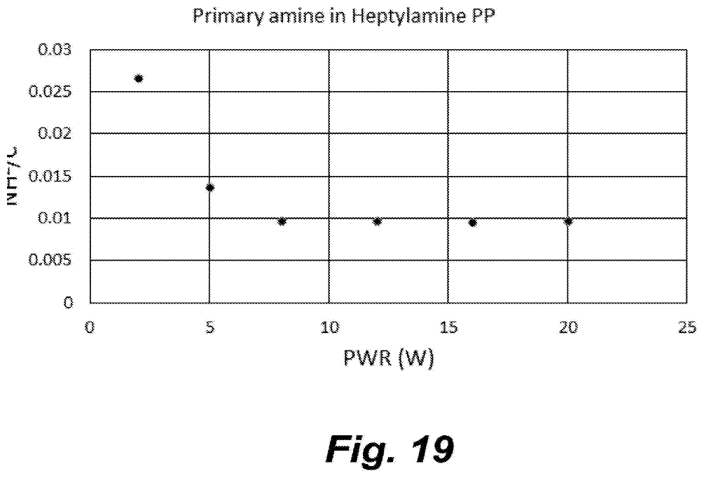

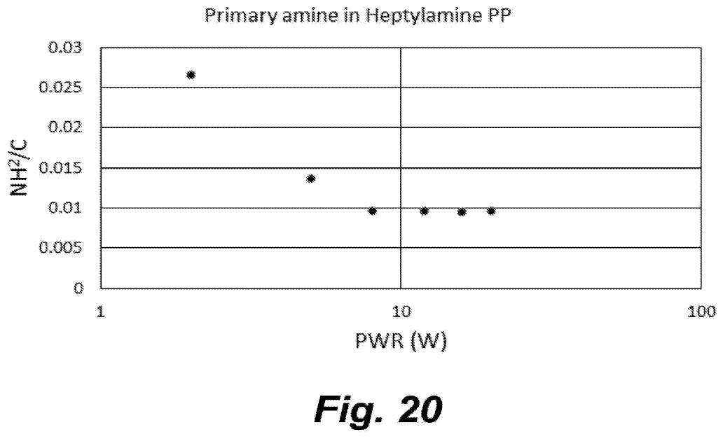

Quantification of primary amines indicated a correlation between plasma power and amine concentration (FIGS. 18 to 20). FIGS. 18 to 20 demonstrate that as power is reduced in the plasma polymerisation process, the relative amount of primary amine increases with respect to both carbon (FIG. 19/20) and nitrogen (FIG. 18).

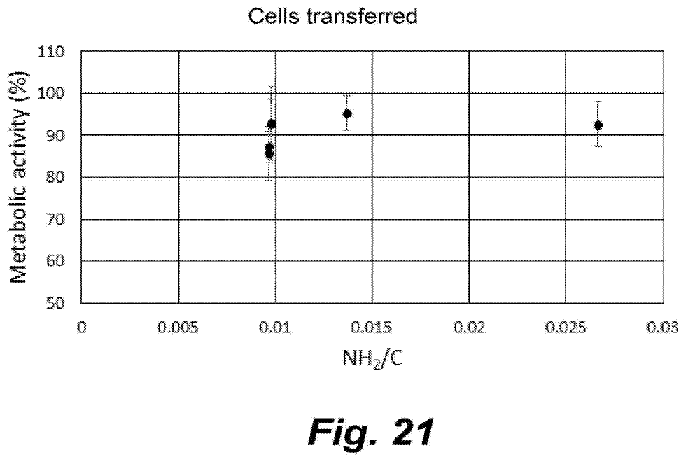

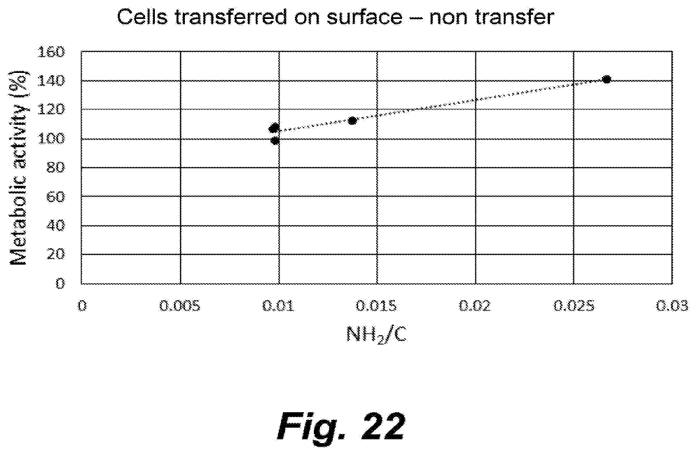

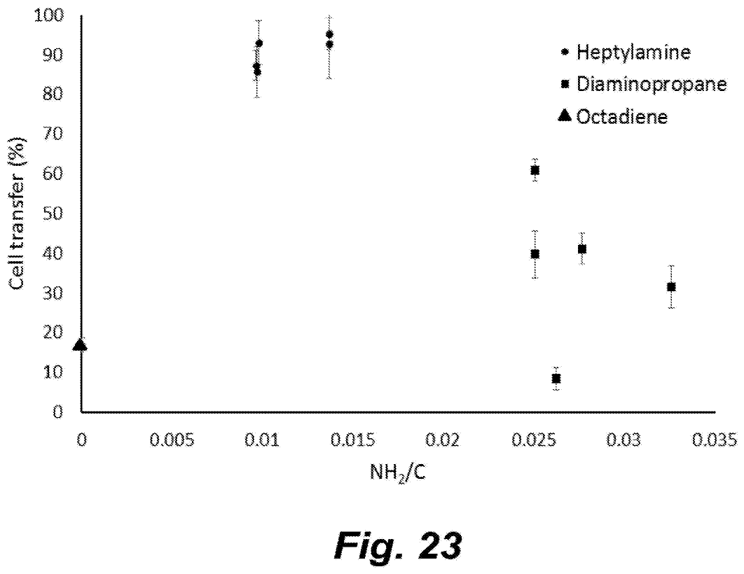

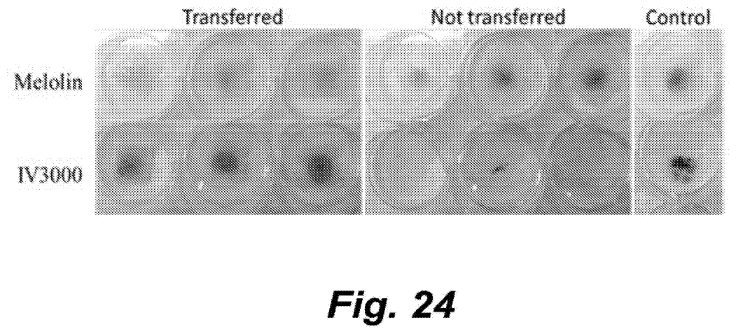

When the amine ratio is plotted against the ability of cells to transfer, this indicates an optimum zone for cell transfer (FIGS. 21 and 22). FIGS. 21 and 22 demonstrate that there is an improved level of primary amine to carbon ratio for cell transfer, with a primary amine to carbon ratio for cell transfer of greater than 0.005 (0.5%) NH₂/C showing improved transfer and an optimum ratio being indicated by the peak of the curve shown in FIGS. 21 and 23 (ie. at 0.014 NH₂/C).

FIG. 22 shows the results of the metabolic activity of non-transferred cells.

FIG. 23 shows the results of cell transfer studies using heptylamine, diaminoproapane or octadiene functionalised medical grade silicone substrate as a function of the primary amine to carbon ratio. Octadiene functionalised substrates (which do not contain primary amine) showed a cell transfer of less than 20%.

The heptylamine functionalised substrate showed improved cell transfer over the octadiene functionalised substrate. Further, the heptylamine functionalised substrates showed efficient cell transfer and had a primary amine to carbon ratio of greater than 0.009.

The diaminopropane functionalised substrate generally showed improved cell transfer over the octadiene functionalised substrate and had a primary amine to carbon ratio of greater than 0.025.

### EXAMPLE 6—PRODUCTION OF AMINE FUNCTIONALISED POLYURETHANE AND ATTACHMENT OF CELLS

The plasma polymerisation reaction was performed as previously described herein and the coated fabric removed and stored as usual.

Cells were transferred as previously described herein. All cell assays were performed in the same way as previously described herein.

### EXAMPLE 7—DELIVERY OF OTHER CELL TYPES AND USE OF DIFFERENT SUBSTRATES

The aim of this study was to further assess the optimised amine plasma polymer coating and deteimine whether this surface on the silicone patch could deliver other cell types. Furthermore, these studies were used to ass whether substrates other than silicone may be used for the delivery of Multipotent Adult Progenitor Cells (MAPCs).

Abbreviations:
MAPC Multipotent Adult Progenitor Cell
PP Plasma polymer

Objectives: 1. To determine whether different backing surfaces (other than silicone) can be PP coated and used to deliver cells. Melolin (perforated polyester) and IV3000 (permeable polyurethane) were tested. 2. To see if the PP treated silicone optimised surface can be used to deliver other cell types.

Methodology

Other substrates. Melolin (obtained from Smith & Nephew) and IV3000 (obtained from Smith & Nephew) were selected and coated with an amine PP substrate as described herein. Melolin is a highly absorbent cotton and acrylic fibre pad which is heat bonded on one side to a very thin perforated polyester film to which cells are to be seeded. IV3000 is a polyurethane dressing.

Cell seeding and transfer assay was then performed as described herein.

Other cell types. For studies using other cell types, silicone with amine PP patches was prepared. Human primary fibroblasts and keratinocytes from multiple donors were seeded and a transfer assay and analysis performed as described herein.

Statistical analysis. Normality and equal variance was assumed. 2-tailed t-test was used for the comparison

Results

Visual inspection of MTT stained cells transferred to dermis is an initial and qualitative measure of cell delivery. The aim was to deliver a homogeneous and regular population of cells with a visual appearance similar to those under normal culture conditions.

We have found that the silicone with amine PP patch is able to deliver MAPC cells. A comparison of amine PP coated Melolin and IV3000 (FIG. 24) revealed that the permeable polyurethane (IV3000) was able to deliver an apparent healthy and dense population of cells to the DED and leave relatively few cells on the dressing. The positive control exhibited irregularity, resulting from handling difficulties. The polyurethane was found to be difficult to handle due to its thinness and propensity to cling to itself in solution. One of the IV3000 dressings retained some cells and retained them in small circles, which correspond to the small holes in the cell grids used to weight the dressing down. These are the areas that were clearly not in intimate contact with the DED.

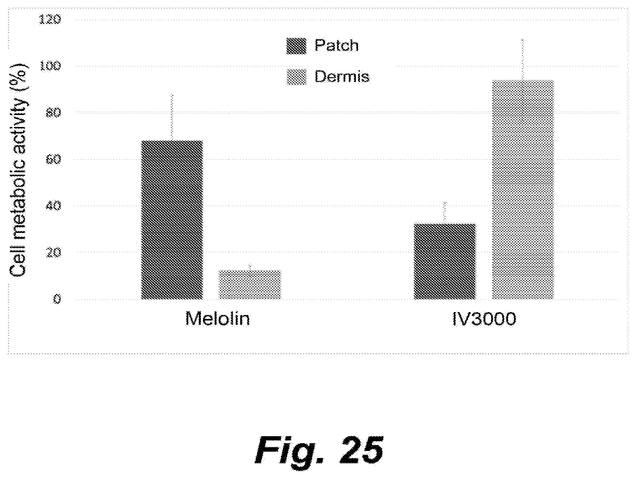

Whilst some cell delivery was evident with perforated polyester (Melolin), it was found to be less effective in the delivery of MAPC cells. The positive control of MAPC cultured on the patch was as expected but the cells did not efficiently migrate from the dressing onto the DED (FIG. 24, not transferred). Elution and quantification of the MTT-formazan product (FIG. 25) supported the conclusions drawn from the images in FIG. 1.

A selection of fibroblasts and keratinocytes each from different donors were assessed. These cells were delivered from the silicone PP surface that was optimised for MAPCs. The non-transferred controls for each cell type were, at the 48 hour time point, not significantly different from the cells cultured on tissue culture plastic (p=9.7), indicating that the optimised amine PP surface was suitable for the culture of both cell types for the duration assessed.

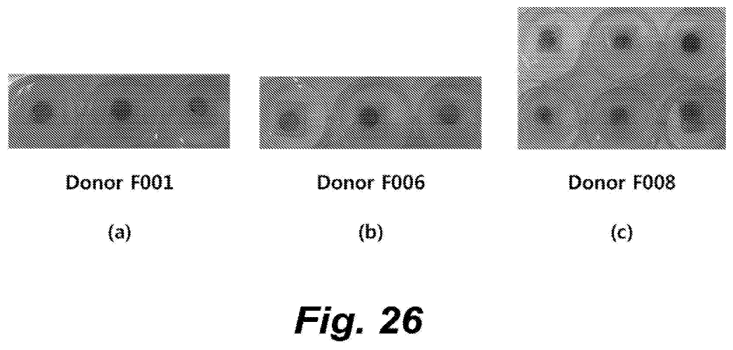

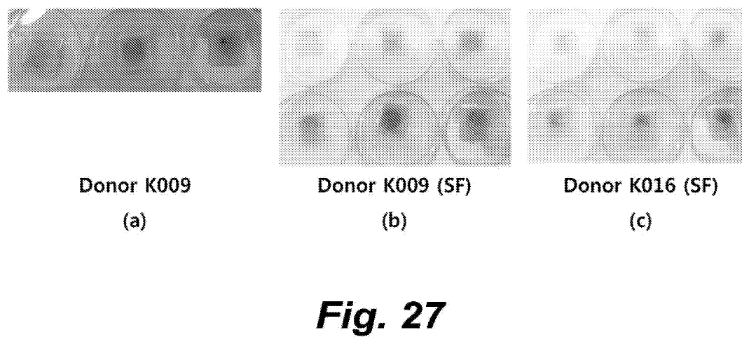

The fibroblasts transferred well to the DED from this surface (FIG. 26) with a quantified delivery in the range 42 to 50% (Table 1). Keratinocytes were less efficiently delivered (FIG. 27) with delivery in the range 14% when grown in a high calcium serum containing medium, Greens Medium to 22% when grown in low calcium serum free conditions (Table 7).

TABLE 7
Quantification of the cell metabolic activity
from delivered cells detailed in FIGS. 26 & 28

| Cells | Delivery (%) | St. dev |
|---|---|---|
| F001 | 50.7 | 8.6 |
| F006 | 49.0 | 7.7 |
| F008 | 42.0 | 13.9 |
| K009 | 14.7 | 4.2 |
| K009 (SF) | 22.7 | 4.9 |
| K016 (SF) | 21.1 | 4.4 |

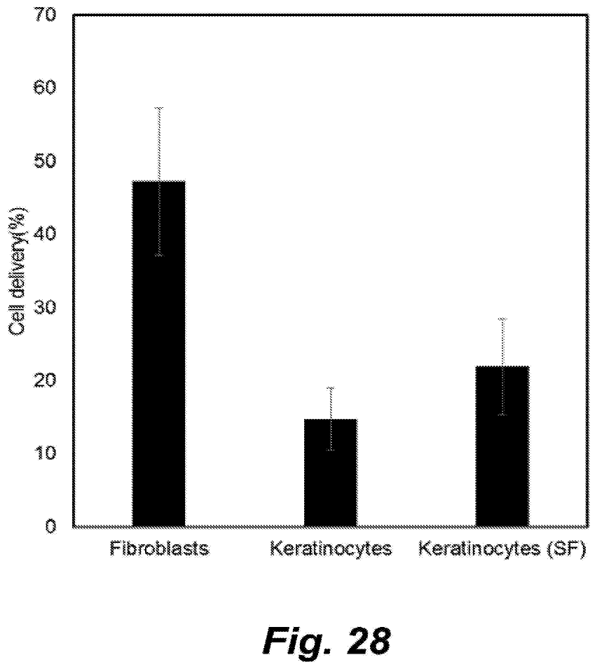

Taken as a whole for each cell type (FIG. 28), it can be seen that both cell types may be delivered from the PP patch, with a significantly higher number of fibroblasts delivered than keratinocytes (p=0.0016).

### EXAMPLE 8—TREATMENT OF WOUNDS IN DIABETIC MICE BY DELIVERY OF MAPC ON PLASMA POLYMER-COATED DRESSING

The administration of cells was investigated in acute wounds in mice. The application of the cells was also compared to where cells were injected into the wound margins of acute wounds in mice. The data shows that the treatments showed a significant improvement when compared to treatments without cells. The administration of cells using the coated polymer showed a significant 22% increase in the rate of healing at day 3 (p<0.001).

The administration of cells via coated polymer patch was also tested using the same protocols in diabetic mouse wounds. A significant improvement in healing (p <0.05) was observed when the cells were delivered to the wounds by the polymer patch vs injection, indicating that delivery of the cells by polymer patch to wounds is a potential therapeutic approach for the treatment of diabetic wounds.

Abbreviations:
HaPP Heptylamine Plasma Polymer
PBS Phosphate Buffered Saline

Methodology

Proprietary multipotent adult progenitor stem cells (MAPCs) were obtained. Using a heptylamine plasma polymer (HaPP)-coated medical grade silicone dressing, the delivery of the cells from the dressing to acute wounds was compared with injection of the cells around the wound site. The HaPP-coated medical grade silicone delivery of cells was then tested in diabetic mouse wounds and compared to an injection of cells around the wound site.

Cells (MAPCs) were cultured and quality control of the harvested cells confirmed they had the correct phenotype, prior to frozen storage under liquid N₂. The cells were thawed, resuspended in sterile PBS, counted using a NucleoCounter and stored on ice until required for the treatments.

Balb/C mice were made diabetic via repeated injection of streptozotocin, which kills the islet cells of the pancreas, rendering the mice incapable of producing sufficient insulin to adequately control their blood glucose levels. The mice were monitored daily and administered insulin as required to maintain their blood glucose levels within the diabetic range. Non-diabetic mice was also used.

The plasma polymer dressing used here comprises an FDA-approved polymer substrate, medical grade silicone; onto which is applied the heptylamine based plasma polymer coating as described herein. For the purposes of the mouse wound studies, HaPP- medical grade silicone dressing with an area of 1 cm² was prepared. Briefly, for each 1 cm² HaPP-PDMS dressing cells were seeded at a density of 20×10³ cells/patch.

During the development of the HaPP- medical grade silicone dressing, it was confirmed that there were no adverse phenotypic and genotypic changes to the cells after culture on the dressing for up to 24 hours.

For surgical procedures the mice were placed under anaesthetic, and two 6 mm excisional wounds were made, via punch biopsy, on the dorsum of each mouse. Initially, a dose response study was carried out treating the wounds with 10×10³, 20×10³, 40×10³ and 80×10³ cells administered using the HaPP- medical grade silicone dressing in diabetic mouse wounds. At day 3 the 20×10³ and 40×10³ MAPC treatments had healed significantly faster than the 80×10³ MAPC treatment. Administration of 20×10³ cells healed significantly faster than all other treatment groups at day 7. This was therefore taken as an optimal dose and used in all other studies.

Three treatments groups were investigated: the first received 20×10¹ cells via polymer patch per wound, the second received intradermal injections of 20×10³ cells per wound and the final group received patch alone. 8 mice were in each group and 3 end points were investigated; at day 7, 10, and 14. Photographs were also taken of the wounds at day 3, 7, 10 and 14 for macroscopic assessment. Wounds were covered with Tegaderm, which was removed after 3 days. This was repeated for the HaPP- medical grade silicone dressing which was also covered with Tegaderm for 3 days and compared to wounds injected with 20×10³ cells in diabetic mice. At the endpoints, wounds were collected, processed, stained and imaged for microscopic measurements.

Results

A dose response study was carried out in diabetic wounds, which were treated with 10×10³, 20×10³, 40×10³ and 80×10³ cells delivered via the HaPP- medical grade silicone patch. It was found that administration of 20×10³ cells healed significantly faster than other treatments by day 7 (10 k, p<0.01; 40 k p<0.04; 80 k p<2×10⁻⁵).

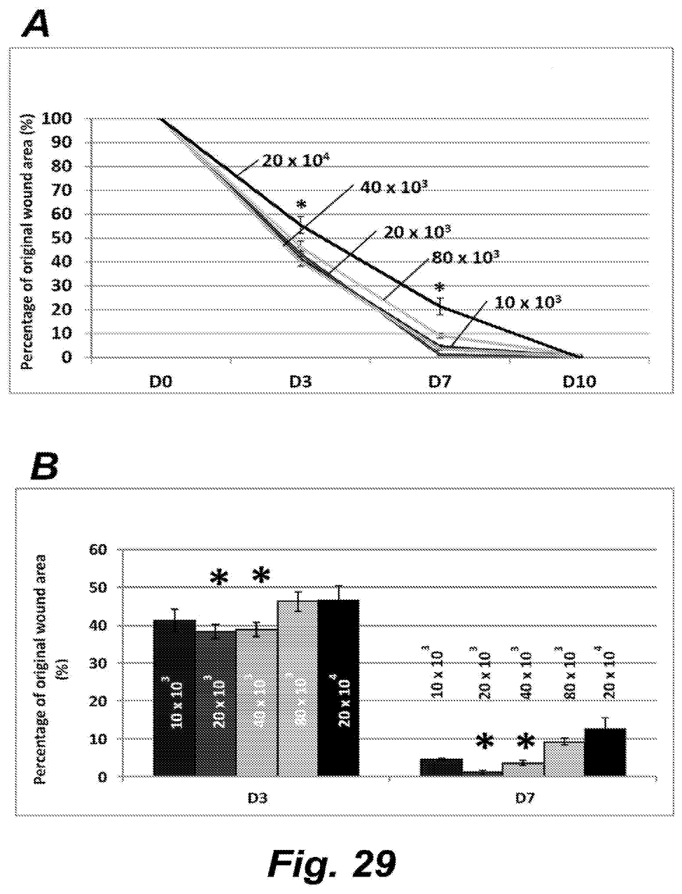

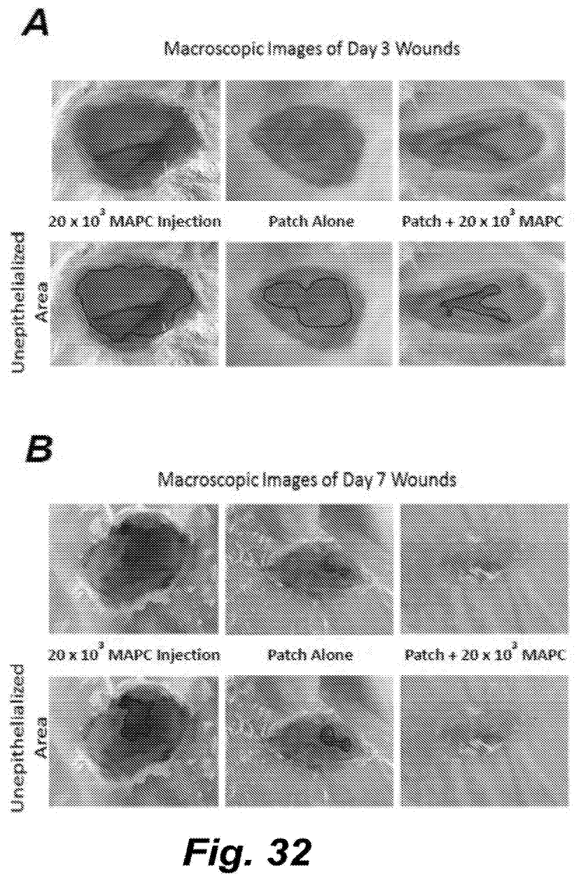

No adverse events were observed and the patches and cells were well tolerated by the mice. The greatest improvement in healing was observed at day 3 when the wound gape was decreased by 32% (FIG. 29A) and when the data was normalised to take into account variation in the initial size of the wound this improvement was 39% (FIG. 29B). Representative images are included in FIG. 32 which shows the increased reepithelialization that occurs with the cells delivered by the HaPP- medical grade silicone patch.

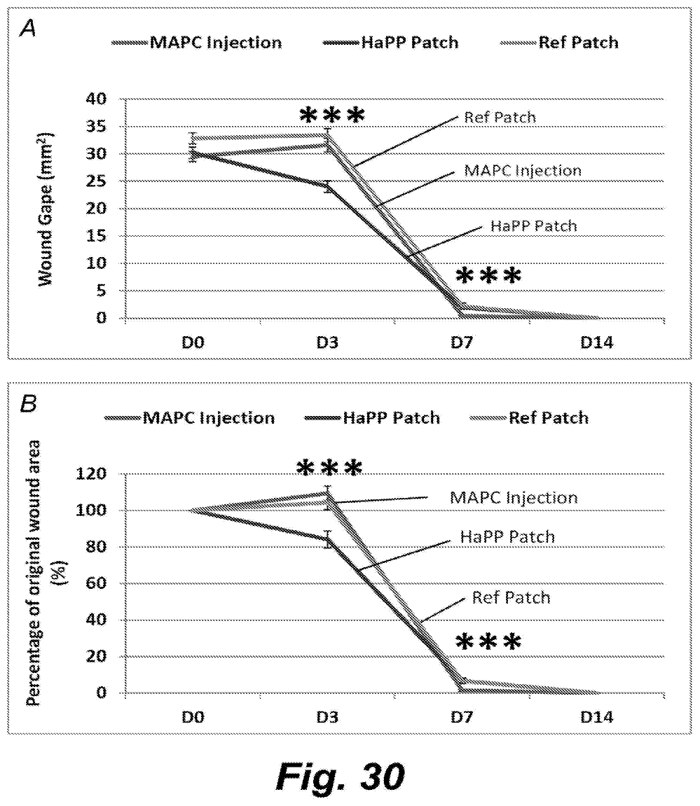

Acute (non-diabetic) wounds treated with 20×10³ cells delivered via any of the the HaPP- medical grade silicone patch, a reference patch, or cells delivered by injection healed significantly faster than those treated with either HaPP-medical grade silicone alone, the reference patch alone or injection of PBS. No adverse events were observed and the patches and cells were well tolerated by the mice. The greatest improvement in healing was observed at day 3 when the wound gape was decreased by 22% (FIG. 30A) and when the data was normalised to take into account variation in the initial size of the wound this improvement was 20% (FIG. 29B).

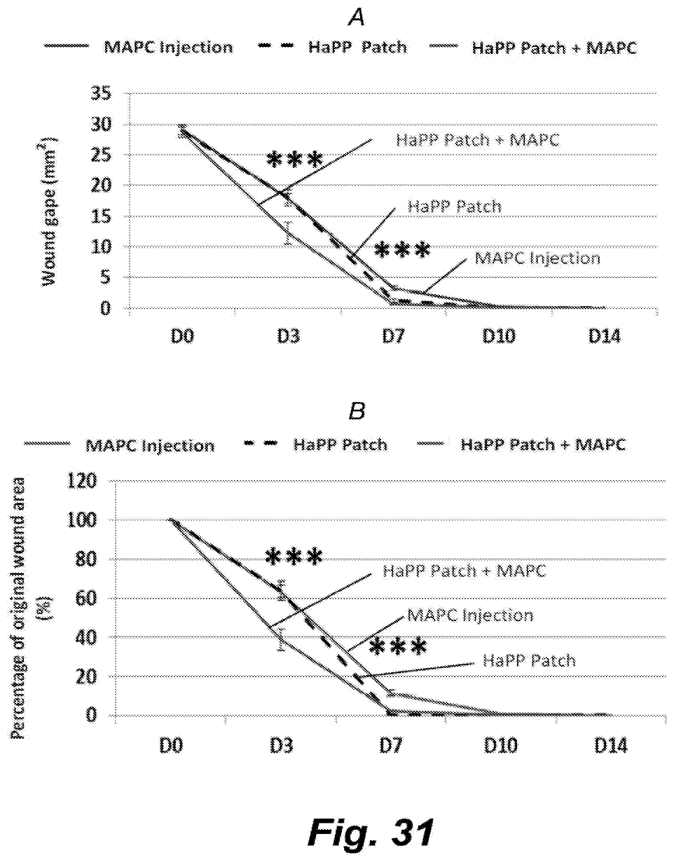

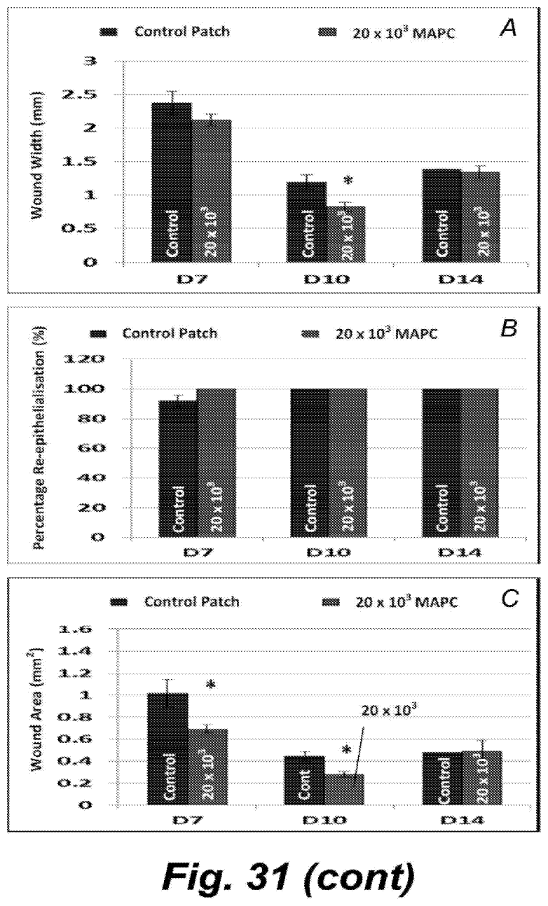

Diabetic wounds treated with 20×10³ cells delivered via the HaPP- medical grade silicone patch healed significantly faster than those treated with either cells delivered by injection or HaPP- medical grade silicone alone. No adverse events were observed and the patches and cells were well tolerated by the mice. The greatest improvement in healing was observed at day 3 when the wound gape was decreased by 32% (FIG. 31 A) and when the data was normalised to take into account variation in the initial size of the wound this improvement was 39% (FIG. 31B). There were also significant reductions seen in wound area at day 7 (32% and p<0.02) and 10 (36% and p<0.005) and wound width at day 10 (31% and p<0.008). Representative images are included in FIG. 32 which shows the increased reepithelialization that occurs with the cells delivered by the HaPP- medical grade silicone patch.

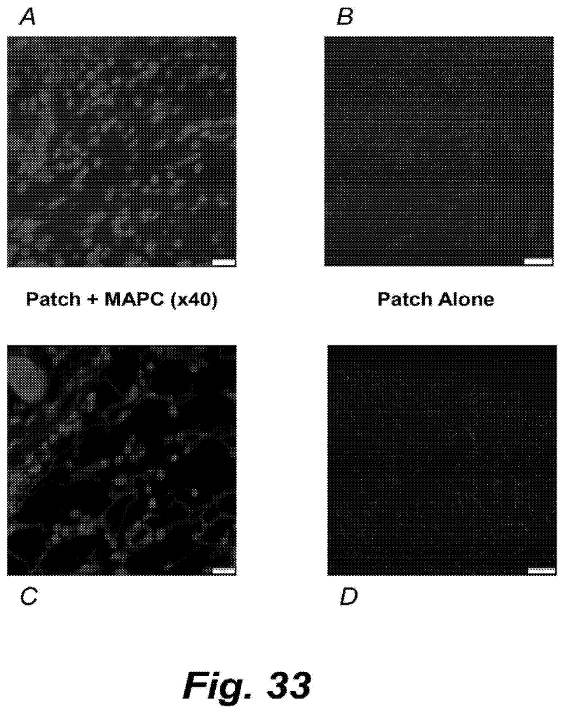

Day 3 and day 7 mouse wounds were also stained with a human nuclear antibody directly labelled with a red fluorescent probe. This antibody therefore detected any human cells embedded in mouse tissue i.e. MAPCs. Numerous cells were stained in the mouse wounds identifying the MAPCs within the wounds. The data is shown in FIG. 33.

### CONCLUSION

In this experiment the efficacy of cells delivered on the HaPP- medical grade silicone dressing was confirmed in the healing impaired, diabetic mouse model. Injection of cells was no more effective in promoting healing than the polymer patch (without cells). However, a significant increase in the rate of healing (p=0.0005), at day 3, was observed when the cells were delivered using the HaPP- medical grade silicone patch (vs. injection) and these wounds healed 32% faster than wounds treated with cells delivered by injection. This increase in the rate of healing was also observed after 7 days where there was a significant increase (p=2.01×10⁻⁵) in the rate of healing in the wounds treated with the cells delivered via the HaPP- medical grade silicone patch.

The data therefore demonstrates that localised delivery from the HaPP- medical grade silicone dressing has benefits over direct or indirect injection of cells on healing outcome and that a reduction in the number of cells required to achieve a therapeutically effect can be achieved.

In conclusion, our studies clearly show that cells delivered using a HaPP-medical grade silicone patch to wounds in diabetic mice promotes rapid and improved healing.

### EXAMPLE 9—TREATMENT OF WOUNDS

To use the patch to heal a foot or leg ulcer, the patch would be placed cell side down onto the wound. It would be secured in place using an appropriate dressing i.e. Tegaderm, and left for a minimum of 24 hours. It is anticipated that the patch could be used to heal wounds such as venous ulcers, ischemic ulcers, neuropathic ulcers and other chronic cutaneous wounds. In some cases, the wound may be in a diabetic subject. The patch may also assist in the healing of other indications such as the healing of a split thickness skin graft on a burns patient. Healing would be assessed by looking at the one or more of the rate of closure of the wound, the rate of reepithelialisation, as well as assessing levels of inflammation and vascularisation of the wound bed. The level of scar formation and wound contraction would also be monitored as indicators of poor wound healing.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications.

The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be understood that all embodiments in this application that are directed to therapeutic compositions for cell delivery, their methods of making, and their methods of use, include attachment of cells to an alkylamine functionalized substrate.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of delivering cells to a biological site, the method comprising:
providing a product comprising a polymer substrate functionalised by plasma polymerisation with an alkylamine to produce an alkylamine functionalised polymer substrate and cells for delivery to the site attached to the alkylamine functionalised polymer substrate, wherein the alkylamine functionalised polymer substrate comprises a surface density with an atomic ratio of primary amine to carbon of greater than 0.005; and
applying the product to the biological site and transferring the cells from the product to the biological site;
thereby delivering the cells to the biological site.

2. The method according to claim 1, wherein the surface density comprises an atomic ratio of primary amine to carbon of 0.005 to 0.035.

3. The method according to claim 1, wherein the surface density comprises an atomic ratio of primary amine to carbon of 0.009 to 0.035.

4. The method according to claim 1, wherein the alkylamine functionalised polymer substrate comprises a substrate functionalised with an alkylamine selected from heptylamine, diaminopropane, or allylamine, and/or a substituted derivative of the any of aforementioned alkylamines.

5. The method according to claim 1, wherein the polymer substrate comprises a silicone, a polyurethane, a nylon, a polyester, a polyethylene, a polypropylene, a polytetrafluoroethylene, a poly(vinylidene fluoride), a polyethylene terephthalate, a polycaprolactone, a rayon, a polydioxanone, a polygalactin, a poly lactic acid, a polyglycolic acid, a poly(glycolide-co-caprolactone), a poly(glycolide-trimethylene carbonate, a polyhydroxybutyrate, a polyhydroxyvalerate, or a copolymer or blend of any of the aforementioned polymers.

6. The method according to claim 1, wherein the biological site comprises a wound and the product comprises a patch, a bandage or a dressing.

7. A method of preventing and/or treating a subject with a disease, condition or state that would benefit from the delivery of cells to the subject, the method comprising delivery of cells to the subject using the method according to claim 1.

8. A method of delivering cells to a biological site, the method comprising:
applying a product to the biological site, the product comprising a polymer substrate functionalised by plasma polymerisation with an alkylamine to produce an alkylamine functionalised polymer substrate and cells for delivery to the site attached to the alkylamine functionalised polymer substrate, wherein the alkylamine functionalised polymer substrate comprises a surface density with an atomic ratio of primary amine to carbon of greater than 0.005; and
transferring the cells from the product to the biological site; thereby delivering the cells to the biological site.

9. The method according to claim 8, wherein the surface density comprises an atomic ratio of primary amine to carbon of 0.005 to 0.035.

10. The method according to claim 8, wherein the surface density comprises an atomic ratio of primary amine to carbon of 0.009 to 0.035.

11. The method according to claim 8, wherein the alkylamine functionalised polymer substrate comprises a substrate functionalised with an alkylamine selected from heptylamine, diaminopropane, or allylamine, and/or a substituted derivative of the any of aforementioned alkylamines.

12. The method according to claim 8, wherein the polymer substrate comprises a silicone, a polyurethane, a nylon, a polyester, a polyethylene, a polypropylene, a polytetrafluoroethylene, a poly(vinylidene fluoride), a polyethylene terephthalate, a polycaprolactone, a rayon, a polydioxanone, a polygalactin, a poly lactic acid, a polyglycolic acid, a poly(glycolide-co-caprolactone), a poly(glycolide-trimethylene carbonate, a polyhydroxybutyrate, a polyhydroxyvalerate, or a copolymer or blend of any of the aforementioned polymers

13. The method according to claim 8, wherein the biological site comprises a wound and the product comprises a patch, a bandage or a dressing.

14. A method of preventing and/or treating a subject with a disease, condition or state that would benefit from the delivery of cells to the subject, the method comprising delivery of cells to the subject using the method according to claim 8.
